(19) United States
(12) Patent Application Publication
(10) Pub. No.: US 2024/0260458 A1
(43) Pub. Date: Aug. 1, 2024

(54) COMPOUND FOR ORGANIC ELECTROLUMINESCENT DEVICE

(71) Applicant: GUANGZHOU CHINARAY OPTOELECTRONIC MATERIALS LTD., Guangzhou, Guangdong (CN)

(72) Inventors: Tao LI, Guangzhou, Guangdong (CN); Zhifei LONG, Guangzhou, Guangdong (CN); Zhijun LONG, Guangzhou, Guangdong (CN); Jingyao SONG, Guangzhou, Guangdong (CN)

(73) Assignee: GUANGZHOU CHINARAY OPTOELECTRONIC MATERIALS LTD., Guangzhou, Guangdong (CN)

(21) Appl. No.: 18/294,125

(22) PCT Filed: Jun. 22, 2021
(86) PCT No.: PCT/CN2021/101554

§ 371 Date: Jan. 31, 2024

(30) Foreign Application Priority Data
Nov. 13, 2020 (CN) 202011270072.X

(51) Int. Cl. H10K 85/60; C07D 263/57; C07D 413/14; C09K 11/06; H10K 50/11

(52) U.S. Cl. CPC H10K 85/657; C07D 263/57; C07D 413/14; C09K 11/06; H10K 50/11; H10K 85/633; H10K 85/636; H10K 85/6572; C09K 2211/1018; H10K 85/622; H10K 85/626; H10K 85/6576

(57) ABSTRACT

The present disclosure relates to an organic compound, a mixture, and an organic electroluminescence device. The compound has a structure as represented by formula (1):

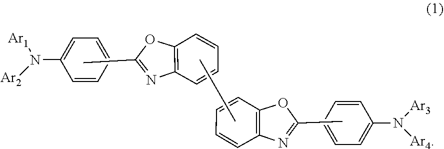

Ar₁ to Ar₄ are independently selected from the group consisting of a substituted or unsubstituted aromatic group having 6 to 40 ring atoms, and a substituted or unsubstituted heteroaromatic group having 5 to 40 ring atoms. Ar₁ and Ar₂ form a ring or do not form a ring; and Ar₃ and Ar₄ form a ring or do not form a ring.

### CROSS REFERENCE

The present disclosure claims priority to Chinese Patent Application No. 202011270072X entitled “A compound for an organic electroluminescent device”, filed on Nov. 13, 2020, the entire contents of which are incorporated herein by reference.

### TECHNICAL FIELD

The present disclosure relates to the field of organic electroluminescence technology, and in particular to a compound for an organic electroluminescent device.

### BACKGROUND

Organic electroluminescent display devices, such as organic light-emitting diodes (OLEDs), are a type of self-luminous display devices. The organic electroluminescent display devices generate excitons through the transfer and recombination of charge carriers between different functional layers. The organic electroluminescent display devices rely on organic compounds or metal complexes with high quantum efficiency to emit light, and have the characteristics of self-luminescence, high brightness, high efficiency, high contrast, and high responsiveness.

In recent years, the luminous efficiency of organic light-emitting diodes (OLEDs) has been greatly improved and their internal quantum efficiency is close to the theoretical limit. Therefore, improving the efficiency of light extraction becomes an effective means to further improve the stability and current efficiency of devices, such as the stacking of metal complexes of the emitting layer and the matching of refractive indices between various functional layers.

For such light-emitting devices, if the light emitted by the light-emitting layer is incident at a certain angle or more into other films, total reflection may occur at the interface between the light-emitting layer and the other films, causing the light emitted by the light-emitting layer to be unable to be emitted from the devices. Therefore, only a part of the emitted light can be utilized. In recent years, in order to improve the efficiency of light extraction, a light-emitting device with a high refractive index “covering layer” has been provided on the outside of a semi-transparent electrode with low refractive index. For example, in 2001, Hung et al. covered a surface of a metal cathode with a layer of approximately 50 nm organic or inorganic compounds to improve the performance of the devices by controlling the thickness and refractive index. In 2003, Riel et al. attempted to evaporate an inorganic compound ZnSe with high refractive index (n=2.6) onto a cathode to improve light extraction efficiency by utilizing the difference in refractive index between functional layers. However, due to the high evaporation temperature and slow evaporation rate of inorganic materials, these compounds have not been widely used in organic electroluminescent devices.

Therefore, a new type of materials for improving the light extraction efficiency of organic electroluminescent devices need to be further developed.

### SUMMARY

In view of the above reasons, one of the main objectives of the present disclosure is to provide a compound for an organic electroluminescent device. The compound can be used as a light extraction layer material in the organic electroluminescent device, to improve the light extraction efficiency of the device. An organic compound with higher refractive indices can be used in the electroluminescent device to improve light extraction efficiency. This type of the compound needs to meet the following conditions: high extinction coefficient in the ultraviolet band (<400 nm) to avoid adverse effects of harmful light on device materials; in the visible light range (>430 nm), the extinction coefficient is close to 0, which has a high transmittance for visible light, to reduce the impact on light output efficiency of the device; the compound has a high refractive index with small difference in the visible light range, and has the characteristics of improving light output and optimizing device structure; the compound has a higher glass transition temperature and thermal decomposition temperature. The technical solutions of the present disclosure are as follows.

A compound for an organic electroluminescent device has a structure as represented by formula (1).

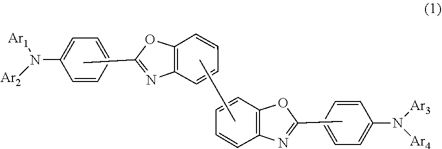

in which,

Ar₁ to Ar₄ are independently selected from the group consisting of a substituted or unsubstituted aromatic group having 6 to 40 ring atoms, and a substituted or unsubstituted heteroaromatic group having 5 to 40 ring atoms.

Ar₁ and Ar₂ form a ring or do not form a ring; and Ar₃ and Ar₄ form a ring or do not form a ring.

A mixture includes the above compound for the organic electroluminescent device and an organic functional material. The organic functional material may be one or more of a hole injection material, a hole transport material, an electron transport material, an electron injection material, an electron blocking material, a hole blocking material, a luminescent material, and a host material.

A composition includes the above compound for the organic electroluminescent device or the above mixture, and at least an organic solvent.

An organic electroluminescent device includes two electrodes, one or more organic functional layers disposed between the two electrodes, and a light extraction layer disposed on a surface of one of the two electrodes away from the organic functional layer. A material of the light extraction layer includes the above compound.

### Beneficial Effects

According to the compound for the organic electroluminescent device of the present disclosure, the compound has a higher glass transition temperature, higher thermal stability, a high extinction coefficient in the ultraviolet band, a smaller extinction coefficient in the visible light range, and a higher refractive index. The compound is capable of being used as a light extraction layer material in the organic electroluminescent device to improve the visible light emission efficiency of the device.

Details of one or more embodiments of the present disclosure are set forth in the accompanying drawings and description below. Other features, objects, and advantages of the present disclosure will be apparent from the description, accompanying drawings, and claims.

### BRIEF DESCRIPTION OF THE DRAWINGS

In order to better describe and illustrate the embodiments or examples of the inventions disclosed herein, reference may be made to one or more accompanying drawings. Additional details or examples used to describe the accompanying drawings should not be considered as limiting the scope of any one of the present disclosure, the currently described embodiments or examples, and the best modes of understanding of these inventions.

FIG. 1 is a structural diagram of a light-emitting device according to an embodiment of the present disclosure. In the figure, 1 is a substrate, 2 is an anode, 3ais a hole injection layer (HIL), 3b is a hole transport layer (HTL), 3c is a light-emitting layer, 3d is an electron transport layer (ETL), 3e is an electron injection layer (EIL), 4 is a cathode, and 5 is a light extraction layer.

FIG. 2 is an ultraviolet visible absorption spectrogram of a compound C33 in a dichloromethane solution.

FIG. 3 is a refractive index and extinction coefficient spectrogram of the compound C33.

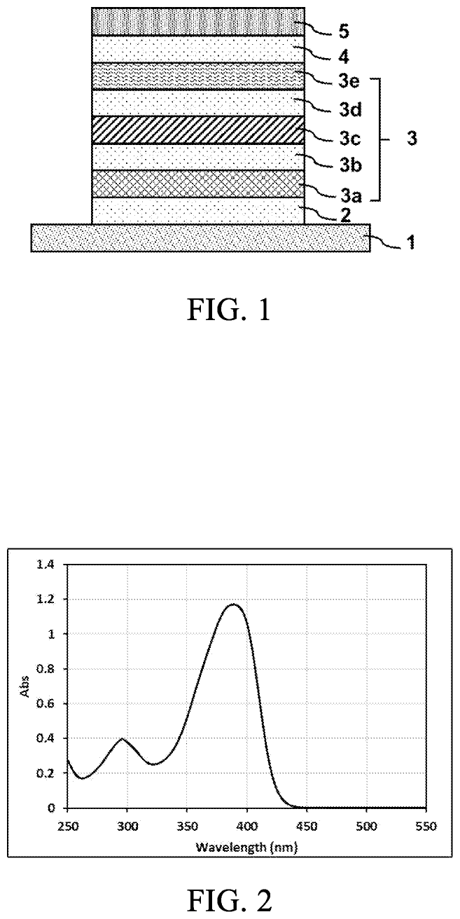

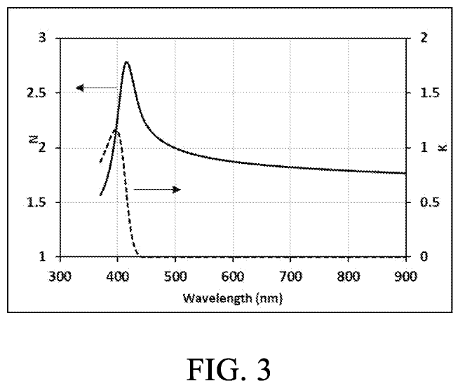

### DETAILED DESCRIPTION

A compound for an organic electroluminescent device is provided by the present disclosure. In order to make the objects, technical solutions, and effects of the present disclosure clearer and more specific, the present disclosure will be further described in detail below. It should be understood that the specific embodiments described herein are merely illustrative of the present disclosure and are not intended to limit the present disclosure.

Unless otherwise defined, all technical and scientific terms used in the context have the same meanings as those commonly understood by those skilled in the art of this field. The terms used in the specification are only for the purpose of describing specific embodiments and are not intended to limit the technical solutions. In the present disclosure, “substitution” means that one or more hydrogen atoms in a group are replaced by one or more substituents.

In the present disclosure, when a same substituent appears more than one times, it can be independently selected from different groups. If a general formula contains a plurality of R₁ groups, R₁ groups may be independently selected from different groups.

In the present disclosure, “substituted or unsubstituted” indicates that a defined group may be substituted or not substituted. When the group is substituted, it should be understood as being optionally substituted by an acceptable group in the art. The acceptable group includes, but not limited to, an C₁₋₃₀ alkyl group, a heterocyclic group containing 3-20 ring atoms, an aryl group containing 5 to 20 ring atoms, a heteroaryl group containing 5 to 20 ring atoms, a silyl group, a carbonyl group, an alkoxycarbonyl group, an aryloxycarbonyl group, an aminoformyl group, a haloformyl group, a formyl group, —NRR′, a cyano group, an isocyano group, an isocyanate group, a thiocyanate group, an isothiocyanate group, a hydroxyl group, a trifluoromethyl group, a nitro group or halogen groups, and the above groups may also be further replaced by acceptable substituents in the art. It is understandable that R and R′ in —NRR′ are independently substituted by acceptable groups in the art, including, but not limited to, H, an C₁₋₆ alkyl group, a cycloalkyl group containing 3 to 8 ring atoms, a heterocyclic group containing 3 to 8 ring atoms, an aryl group containing 5 to 20 ring atoms or a heteroaryl group containing 5 to 10 ring atoms. The C₁₋₆ alkyl group, the cycloalkyl group containing 3 to 8 ring atoms, the heterocyclic group containing 3 to 8 ring atoms, the aryl group containing 5 to 20 ring atoms or the heteroaryl group containing 5 to 10 ring atoms are optionally further substituted by one or more of the following groups: an alkyl group containing 1 to 6 carbon atoms, a cycloalkyl group containing 3 to 8 ring atoms, a heterocyclic group containing 3-8 ring atoms, halogen groups, a hydroxyl group, a nitro group and an amino group.

In the present disclosure, the term “number of ring atoms” represents the number of atoms constituting the ring itself in the compounds (such as monocyclic compounds, fused ring compounds, cross-linked compounds, carbocyclic compounds, and heterocyclic compounds) obtained by atomic bond synthesis. When one or more of the ring atoms are substituted by a substituent, the ring atoms contained in the substituent are not included in the ring atoms. The term “number of ring atoms” described in the context is the same without special explanation. For example, the number of ring atoms of benzene ring is 6, the number of ring atoms of naphthalene ring is 10, and the number of ring atoms of thiophene group is 5.

In the present disclosure, the term “alkyl group” may refer to a straight chain group, a branched chain group, and/or a cyclic alkyl group. The number of carbon atoms of the alkyl group may be 1 to 50, 1 to 30, 1 to 20, 1 to 10, or 1 to 6. Phrases containing the term, such as “C₁₋₉ alkyl group”, refer to alkyl groups containing 1 to 9 carbon atoms. At each occurrence, the C₁₋₉ alkyl group is independently C₁ alkyl, C₂ alkyl, C₃ alkyl, C₄ alkyl, C₅ alkyl, C₆ alkyl, C₇ alkyl, C₈ alkyl, or C₉ alkyl. Non limiting examples of the alkyl group includes methyl, ethyl, n-propyl, isopropyl, n-butyl, sec-butyl, tert butyl, isobutyl, 2-ethyl butyl, 3,3-dimethyl butyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butyhexyl, cyclohexyl, 4-methylcyclohexyl, 4-tert-butylcyclohexyl, n-heptyl, 1-methylheptyl, 2,2-dimethylheptyl, 2-ethylheptyl, 2-butyheptyl, n-octyl, tert octyl, 2-ethyloctyl, 2-butyl octyl, 2-hexyl octyl, 3,7-dimethyloctyl, cyclooctyl, n-nonyl, n-decane, adamantane, 2-ethyldecane, 2-butydecane, 2-hexyldecane, 2-octyldecane, n-undecyl, n-dodecyl, 2-ethyldodecyl 2-Butyldodecyl, 2-hexyldodecyl, 2-octyldodecyl, n-tridecyl, n-tetradecyl, n-pentadecyl, n-hexadecyl, 2-ethylhexadecyl, 2-butyl hexadecyl, 2-hexylhexadecyl, 2-octylhexadecyl, n-heptadecyl, n-octadecyl, n-undecyl, n-eicosyl, 2-ethyleicosyl, 2-butyl eicosyl, 2-hexyleicosyl, 2-octyleicosyl, n-heneicosyl, n-docosalkyl, n-tricosyl, n-lignoceroyl, n-pentacosyl, n-ceryl, n-heptacosyl, n-octacosyl, n-nonascosyl, n-myricyl, adamantane, or the like.

The term “aromatic group” refers to a hydrocarbonyl group that contains at least one aryl ring. The term “heteroaromatic group” refers to an aromatic hydrocarbon group that contains at least one heteroatom. The heteroatom is preferably selected from the group consisting of Si, N, P, O, S, and Ge, particularly preferably selected from the group consisting of Si, N, P, O, and S. A condensed ringaromatic group refers to an aromatic group whose ring may have two or more rings, where two carbon atoms are shared by two adjacent rings, known as a condensed ring. A condensed heterocycle aromatic group refers to a condensed heterocyclic aromatic hydrocarbon group containing at least one heteroatom. For the purpose of the present disclosure, an aromatic group and a heteroaromatic group include not only aryl ring systems, but also non-aryl ring systems. Therefore, a compound having rings such as pyridine, thiophene, pyrrole, pyrazole, triazole, imidazole, oxazole, oxadiazole, thiazole, tetrazole, pyrazine, pyridazine, pyrimidine, triazine, carbene, or the like are considered as the aromatic group or the heterocyclic aromatic group for the purpose of the present disclosure. For the purpose of the present disclosure, a condensed aromatic ring system or a fused heterocycle aromatic system include not only systems with aromatic or heteroaromatic groups, but also a plurality of aromatic or heterocyclic aromatic groups that may be interrupted by short non-aryl units (<10% of non-H atoms, preferably less than 5% of non H atoms, such as C, N, or O atoms). Therefore, systems such as 9,9′-spirodifluorene, 9,9-diarylfluorene, triarylamines, diarylethers, or the like. are also considered as fused aromatic ring systems for the purpose of the present disclosure.

In a preferred embodiment, the aromatic group is selected from the group consisting of benzene, naphthalene, anthracene, fluoranthene, phenanthrene, benzophenanthrene, perylene, tetracene, pyrene, benzopyrene, acenaphthene, fluorene, and derivatives thereof; and the heteroaroaromatic group is selected from the group consisting of triazine, pyridine, pyrimidine, imidazole, furan, thiophene, benzofuran, benzothiophene, indole, carbazole, pyrrolo[1,2-a]imidazole, pyrrolopyrrole, thiophenopyrrole, thienothiophene, furanopyrrole, bis-thf structure, thienofuran, benzisoxazole, benzisothiazole, benzimidazole, quinoline, isoquinoline, cinnoline, quinoxaline, phenanthridine, perimidine, quinazoline, quinazolinone, dibenzothiophene, dibenzofuran, carbazole and derivatives thereof.

The term “amino group” refers to a derivative of an amine with the structural characteristics of the formula —N(X)₂, where each “X” is independently H, a substituted or unsubstituted alkyl group, a substituted or unsubstituted cycloalkyl group, a substituted or unsubstituted heterocyclic group, or the like. The non-limiting examples of the amino group include —NH₂, —N(alkyl)₂, —NH(alkyl), —N(cycloalkyl)₂, —NH(cycloalkyl), —N(heterocyclic)₂, —NH(heterocyclic), —N(aryl)₂, —NH(aryl), —N(alkyl)(aryl), —N(alkyl) (heterocyclic), —N(cycloalkyl)(heterocyclic), N(aryl)(heteroaryl), —N(alkyl) (heteroaryl), or the like.

A center symmetry structure refers to a compound that is completely overlapping when it rotates 180 degrees around the center.

In the present disclosure, the “#” connected to a single bond represents a connecting site or fused sites.

In the present disclosure, when no connecting site is specified in the group, it means that any linkable site in the group may serve as the connecting site;

In the present disclosure, when no fused site is specified in the group, it means that any fused site may be used as the fused site in the group, and two or more adjacent sites in the group are preferred as the fused sites;

In the present disclosure, the “light extraction layer” is located on a surface of an electrode of an organic electroluminescent device away from the organic functional layer, preferably on the surface of the cathode.

A compound for organic electroluminescent devices has a structure as shown in formula (1):

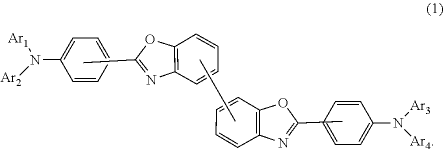

in which:

Ar₁ to Ar₄ are independently selected from the group consisting of a substituted or unsubstituted aromatic group having 6 to 40 ring atoms, and a substituted or unsubstituted heteroaromatic group having 5 to 40 ring atoms.

Ar₁ and Ar₂ form a ring or do not form a ring. Ar₃ and Ar₄ form a ring or do not form a ring.

When Ar₁ and Ar₂ form a ring, the formed formula is represented as follows:

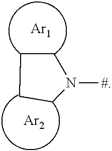

When Ar₃ and Ar₄ form a ring, the formed formula is represented as follows:

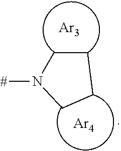

In an embodiment, Ar₁ to Ar₄ are independently selected from the group consisting of a substituted or unsubstituted aromatic group having 6 to 25 ring atoms, and a substituted or unsubstituted heteroaromatic group having 5 to 25 ring atoms.

Preferably, the substituent is selected from the group consisting of an alkyl group having 1 to 30 carbon atoms, an aryl group having 5 to 20 ring atoms, and a heteroaryl group having 5 to 20 ring atoms. Furthermore, the substituents are selected from the group consisting of an alkyl group having 1 to 6 carbon atoms, an aryl group having 5 to 20 ring atoms, and a heteroaryl group having 5 to 10 ring atoms.

In an embodiment, the formula (1) is selected from the following structure:

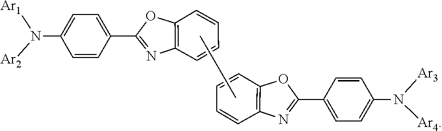

In an embodiment, the organic compound has a center symmetric structure.

Preferably,

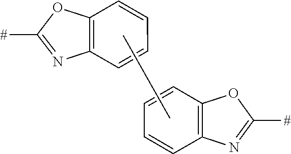

selected from the group consisting of the following structure.

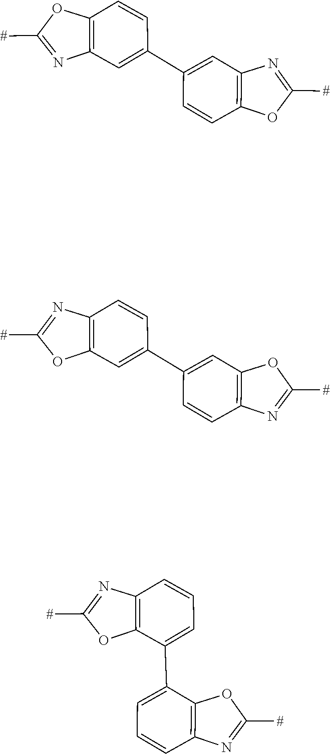

In which, “#” represents the connecting site.

In an embodiment, the compound for the organic electroluminescent device has a structure as represented by formula (2-1) or formula (2-2).

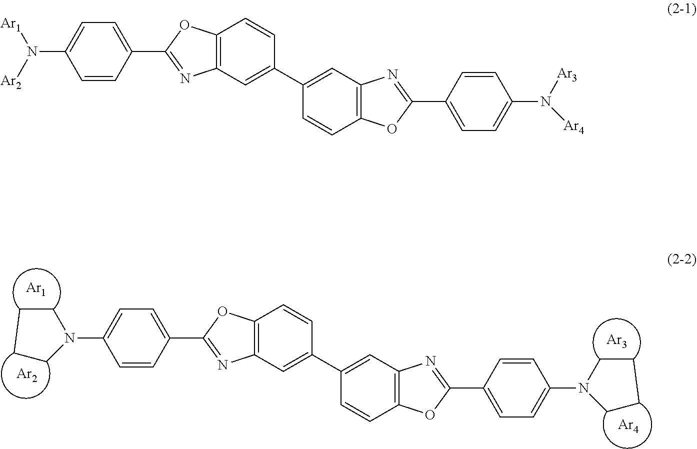

In an embodiment, the compound for the organic electroluminescent device has a structure as represented by any one of general formulae (2-3) to (2-6).

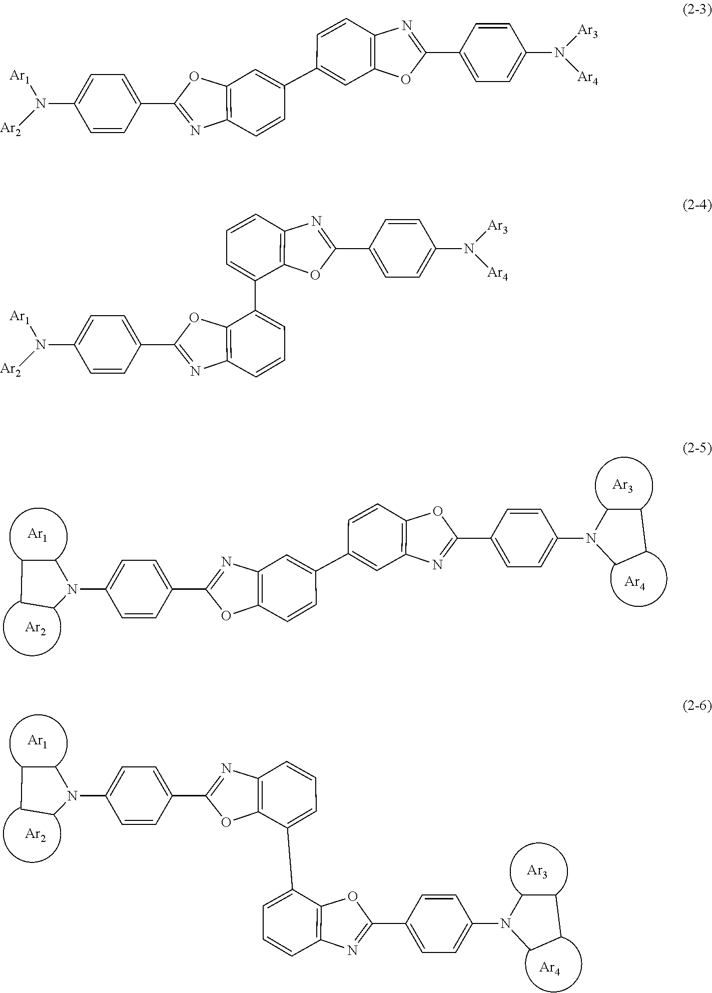

In which, in the formulae (2-1), (2-3), and (2-4), Ar₁ and Ar₂ do not from a ring, and Ar₃ and Ar₄ do not from a ring.

Preferably, the organic compound is selected from the formula (2-1) or (2-2).

In an embodiment, Ar₁ to Ar₄ are independently selected from the group consisting of the following groups.

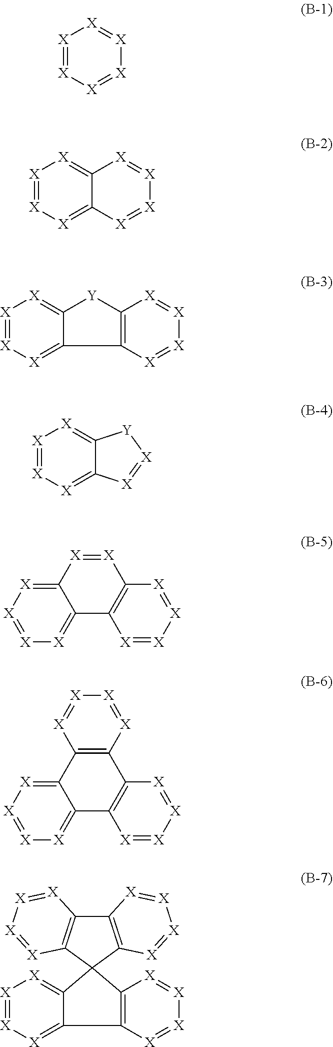

in which:

at each occurrence, X is independently selected from the group consisting of CR₁ and N.

At each occurrence, Y is independently selected from the group consisting of O, S, NR₂, and CR₃R₄.

At each occurrence, R₁ to R₄ are independently selected from the group consisting of —H, -D, a linear alkyl group having 1 to 20 carbon atoms, a linear alkoxy group having 1 to 20 carbon atoms, a linear thioalkoxy group having 1 to 20 carbon atoms, a branched or cyclic alkyl group having 3 to 20 carbon atoms, a branched or cyclic alkoxy group having 3 to 20 carbon atoms, a branched or cyclic thioalkoxy group having 3 to 20 carbon atoms, a silyl group, a ketone group having 1 to 20 carbon atoms, an alkoxycarbonyl group having 2 to 20 carbon atoms, and an aryloxycarbonyl group having 7 to 20 carbon atoms, a cyano group, a carbamoyl group, a haloformyl group, a formyl group, an isocyano group, an isocyanate group, an thiocyanate group, an isothiocyanate group, a hydroxyl, a nitro group, amino, —CF₃, —Cl, —Br, —F, a crosslinkable group, a substituted or unsubstituted aromatic group having 5 to 60 ring atoms, a substituted or unsubstituted heteroaryl group having 5 to 60 ring atoms, an aryloxy group having 5 to 60 ring atoms, a heteroaryloxy group having 5 to 60 ring atoms, and any combination of these groups.

When X is a connecting site or a fused site, X is C.

Furthermore, at each occurrence, R₁ to R₄ are independently selected from the group consisting of —H, -D, a linear alkyl group having 1 to 10 carbon atoms, a branched or cyclic alkyl group having 3 to 10 carbon atoms, a cyano group, a nitro group, —CF₃, —Cl, —Br, —F, a substituted or unsubstituted aromatic group having 5 to 30 ring atoms, and a substituted unsubstituted heteroaromatic group having 5 to 30 ring atoms, and any combination of the groups.

Furthermore, at each occurrence, R₁ to R₄ are independently selected from the group consisting of H, methyl, tert-butyl, phenyl, naphthyl and pyridinyl and any combination of the groups.

Furthermore, Ar₁ to Ar₄ are independently selected from the group consisting of phenyl, biphenyl, terphenyl, naphthyl, phenanthryl, a triphenylenyl group, a spirocyclic group, pyridinyl, pyrimidinyl, triazinyl, quinolyl, isoquinolyl, dibenzofuranyl, dibenzothiophenyl, fluorenyl, 9,9-dimethylfluorenyl, carbazolyl, 9-phenyl-9 carbazolyl, benzofuranyl, benzothiophnyl, benzoxazolyl, benzothiazolyl, and any combination of the groups.

In some embodiments, when the compound is selected from the group consisting of the formula (2-1), (2-3), and (2-4), Ar₁ to Ar₄ are independently selected from the group consisting of following groups.

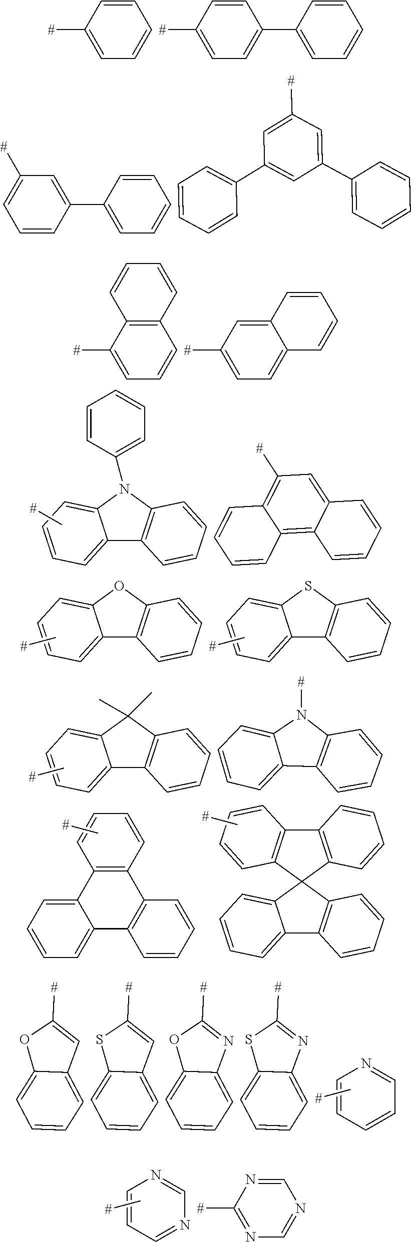

In which, “#” represents the connecting site.

In an embodiment, when the compound is selected from the formulae (2-2), (2-5), or (2-6), Ar₁ to Ar₄ are independently selected from the group consisting of following groups.

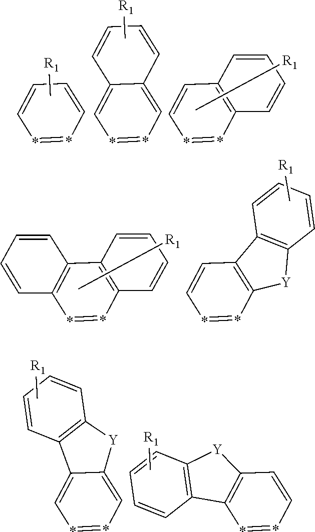

In which, “*” represents the fused sites.

In an embodiment, when the compound is selected from the formula (2-2), Ar₁ to Ar₄ are independently selected from the group consisting of the following groups:

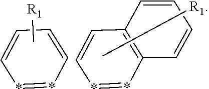

In an embodiment, Ar₁ and Ar₄ are selected from

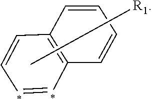

The compound for the organic electroluminescent device according to the present disclosure includes, but not limited to, the following structures.

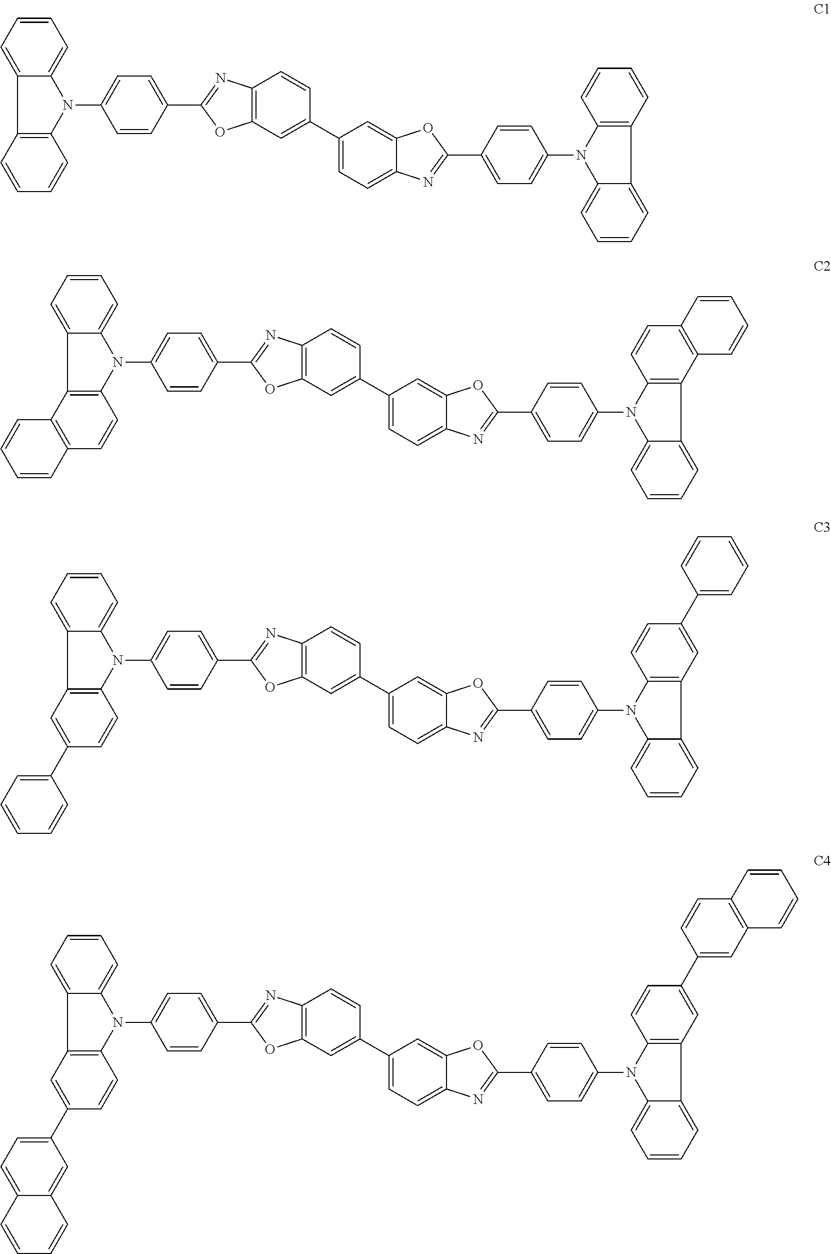

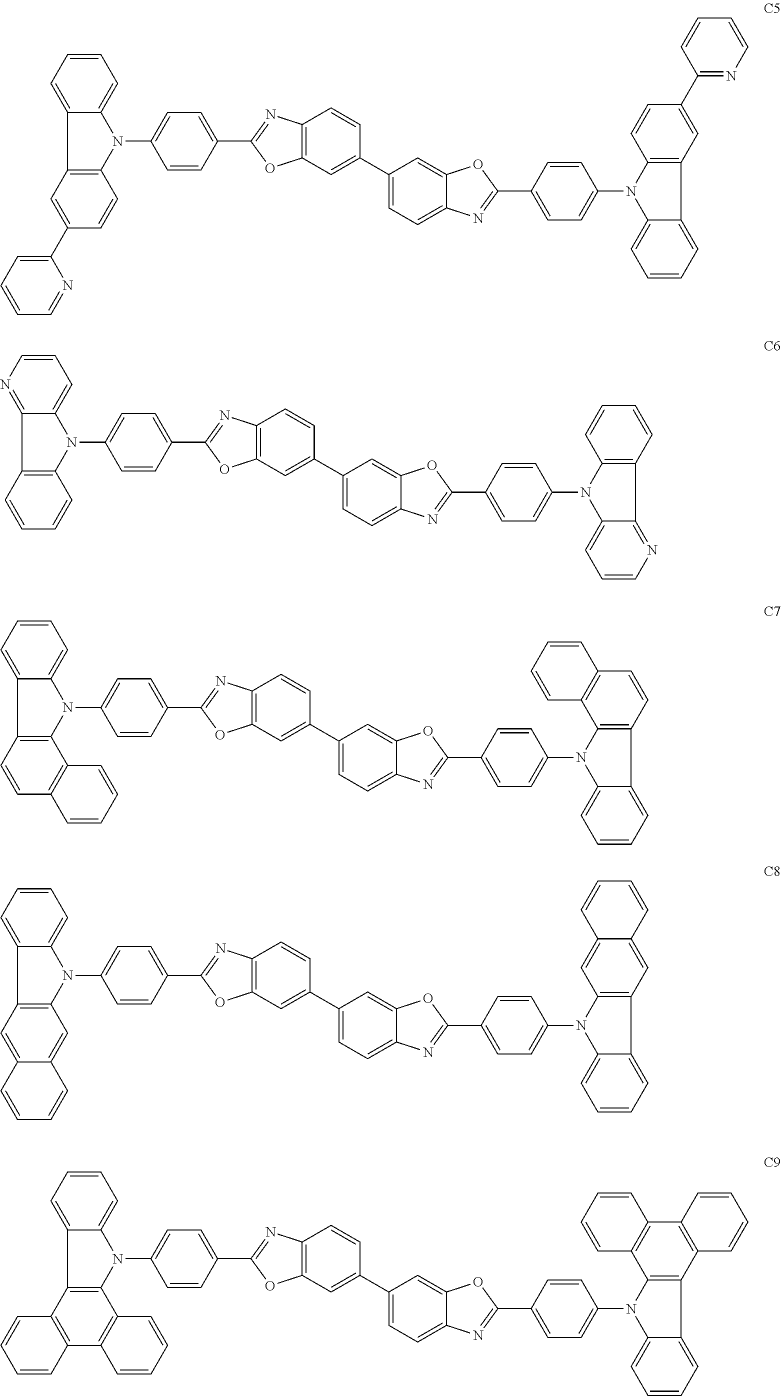

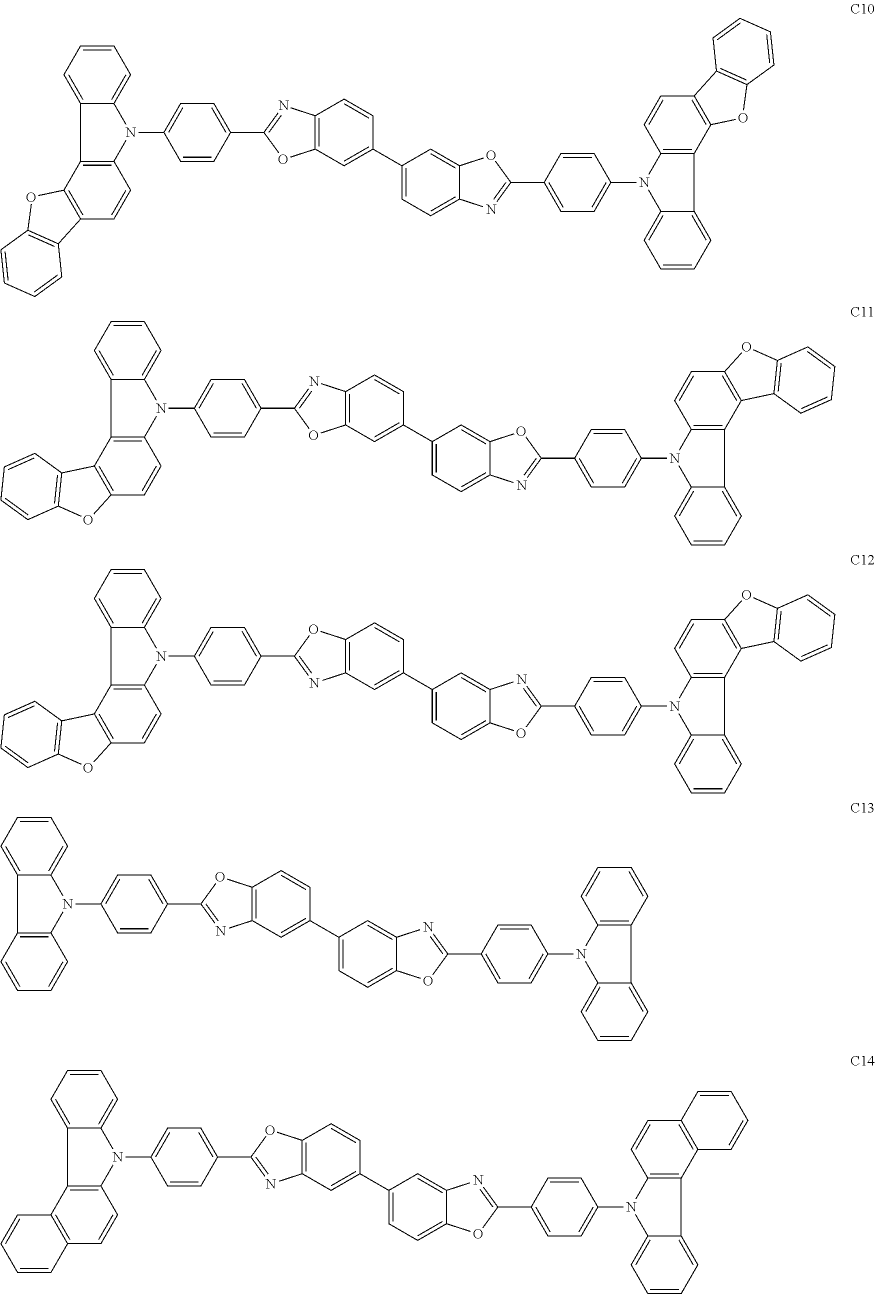

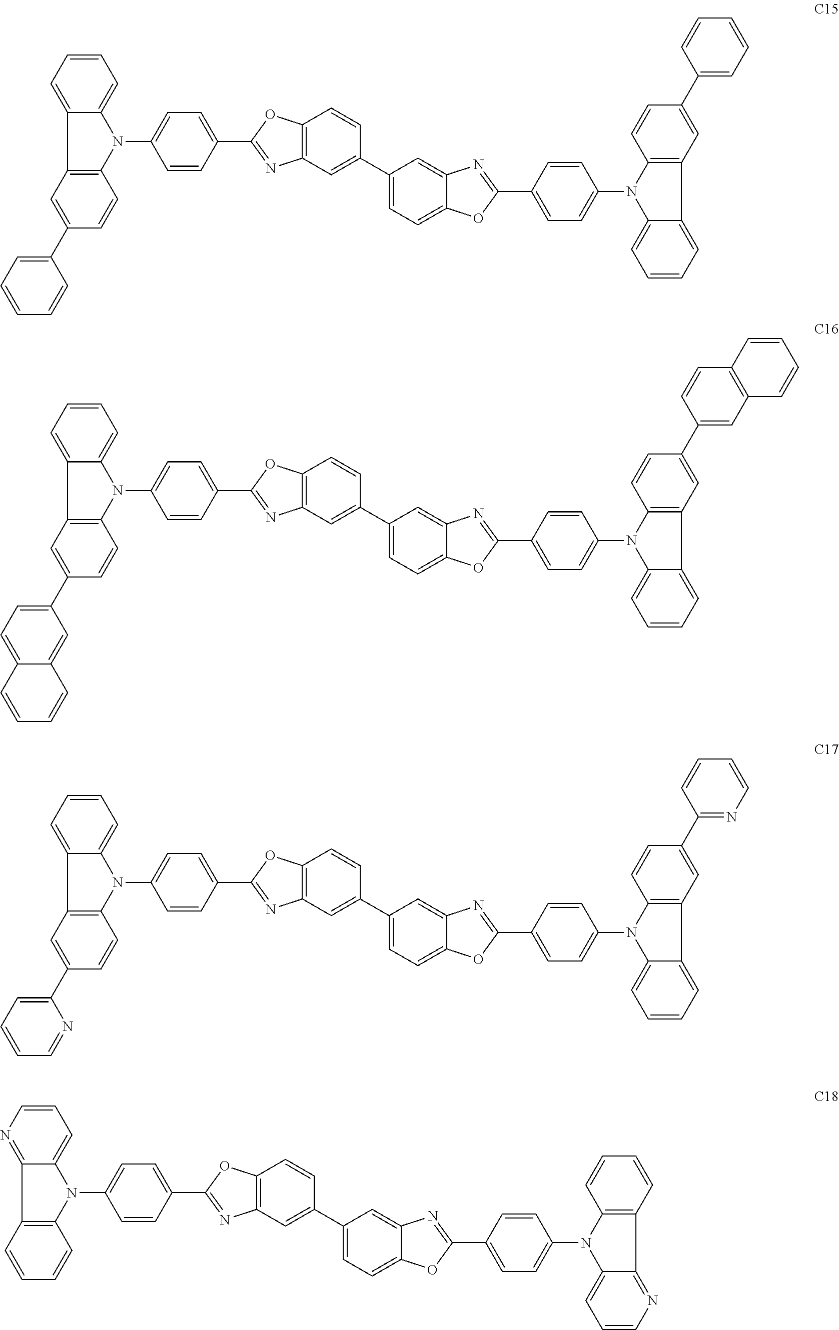 [SMILES: c1ccc(-c2ccc3c(c2)c2ccccc2n3-c2ccc(-c3nc4cc(-c5ccc6oc(-c7ccc(-n8c9ccccc9c9cc(-c%10ccccc%10)ccc98)cc7)nc6c5)ccc4o3)cc2)cc1 ; c1ccc2cc(-c3ccc4c(c3)c3ccccc3n4-c3ccc(-c4nc5cc(-c6ccc7oc(-c8ccc(-n9c%10ccccc%10c%10cc(-c%11ccc%12ccccc%12c%11)ccc%109)cc8)nc7c6)ccc5o4)cc3)ccc2c1 ; c1ccc(-c2ccc3c(c2)c2ccccc2n3-c2ccc(-c3nc4cc(-c5ccc6oc(-c7ccc(-n8c9ccccc9c9cc(-c%10ccccn%10)ccc98)cc7)nc6c5)ccc4o3)cc2)nc1 ; c1ccc2c(c1)c1ncccc1n2-c1ccc(-c2nc3cc(-c4ccc5oc(-c6ccc(-n7c8ccccc8c8ncccc87)cc6)nc5c4)ccc3o2)cc1]

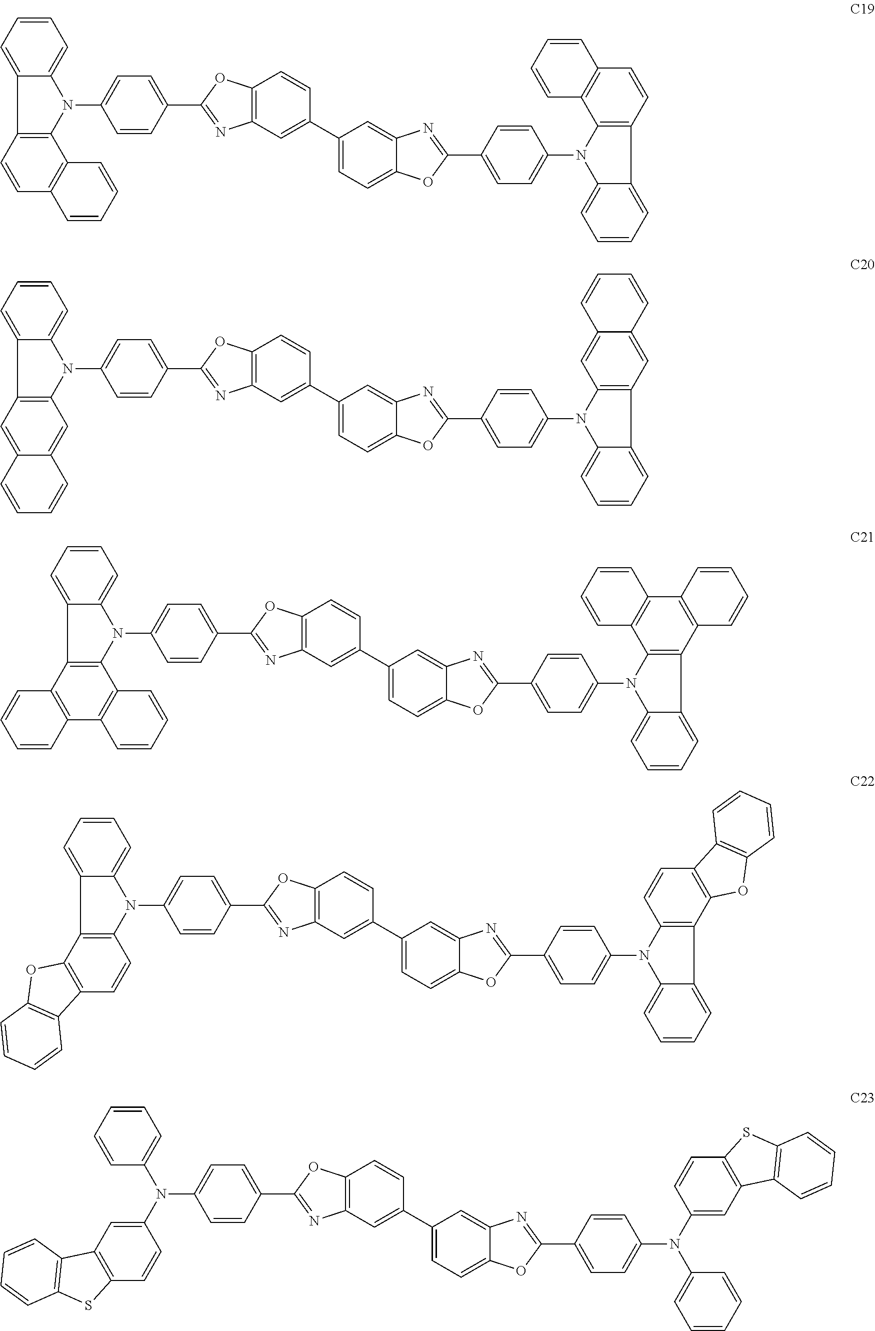

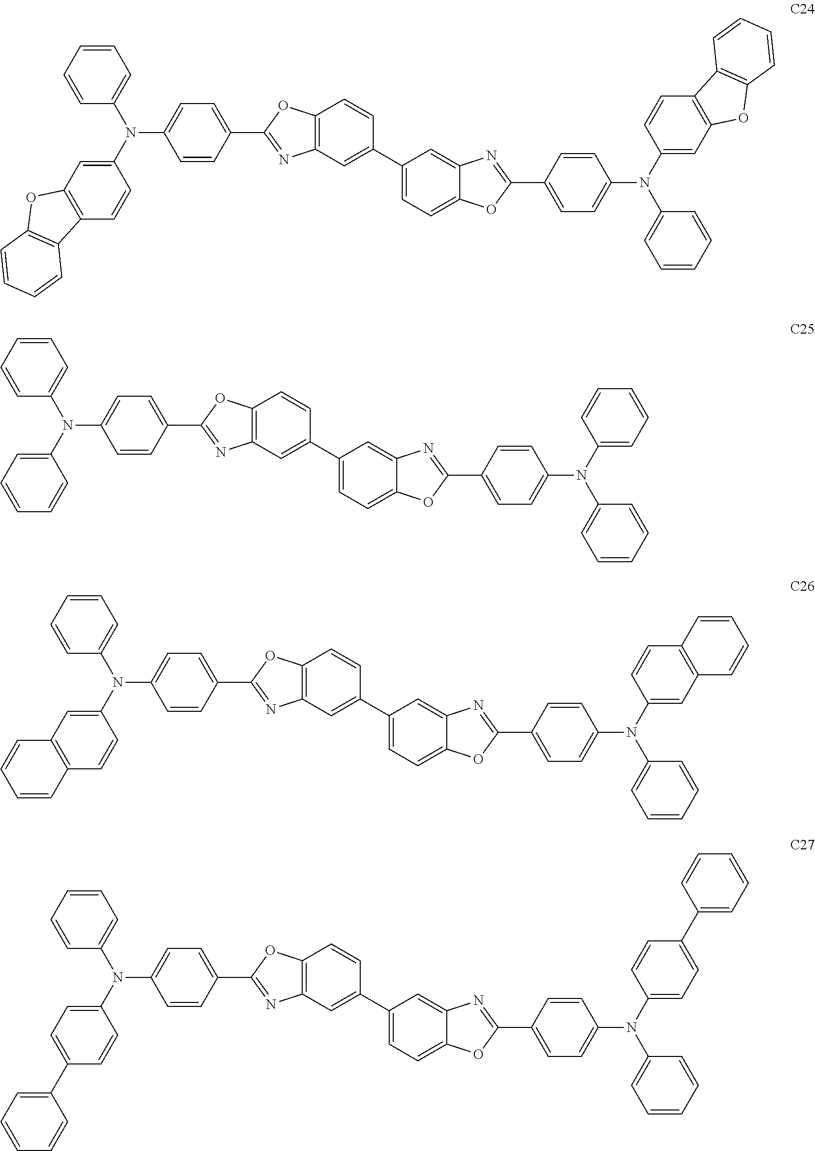

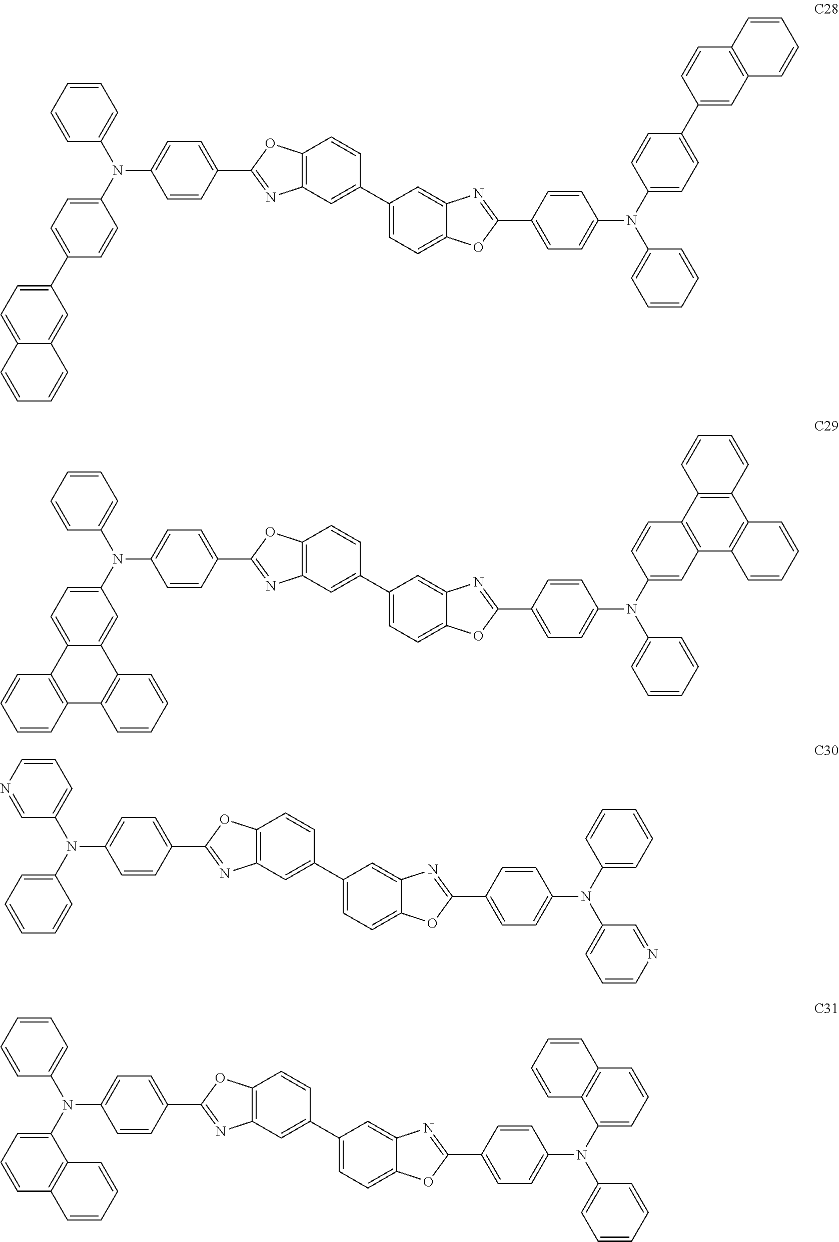

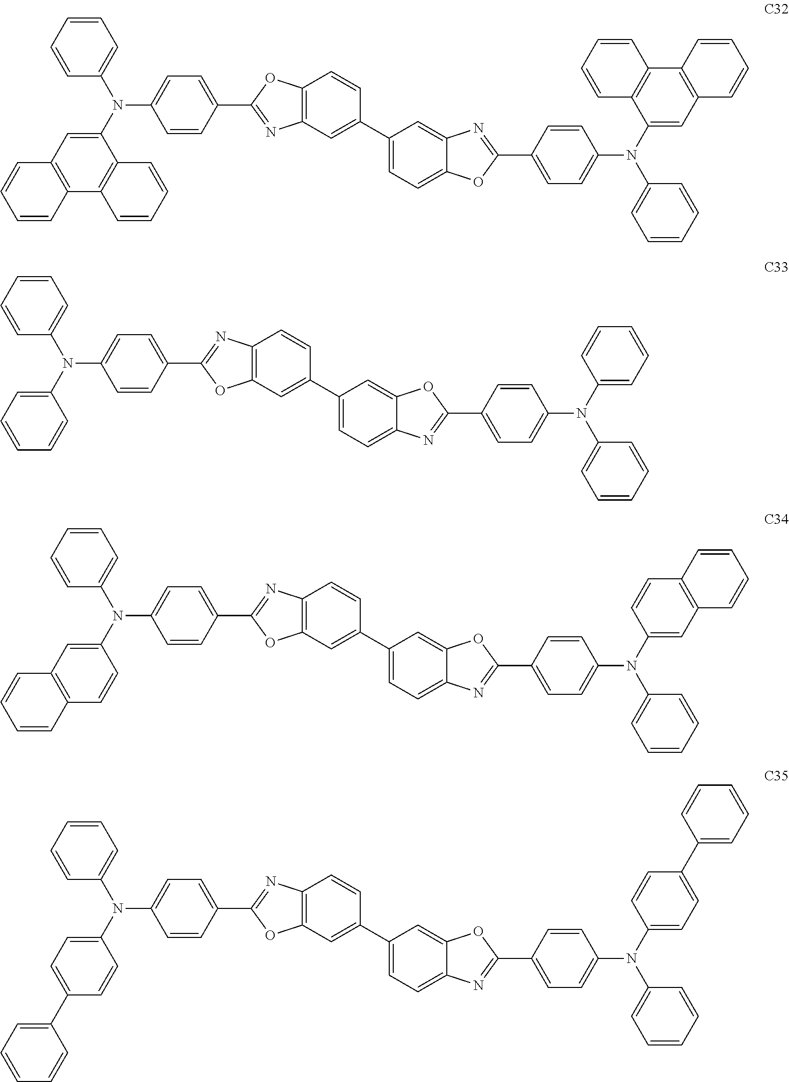

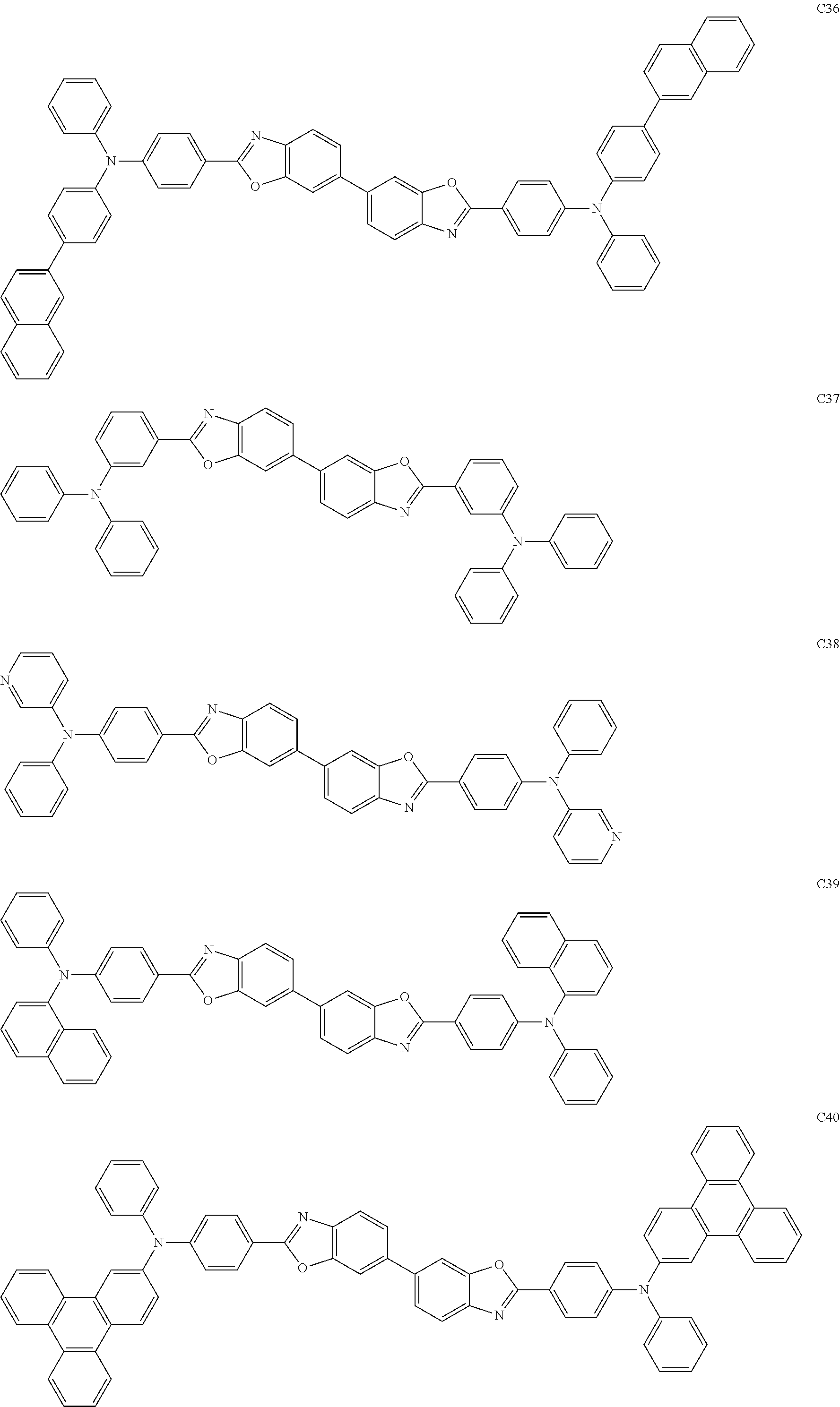

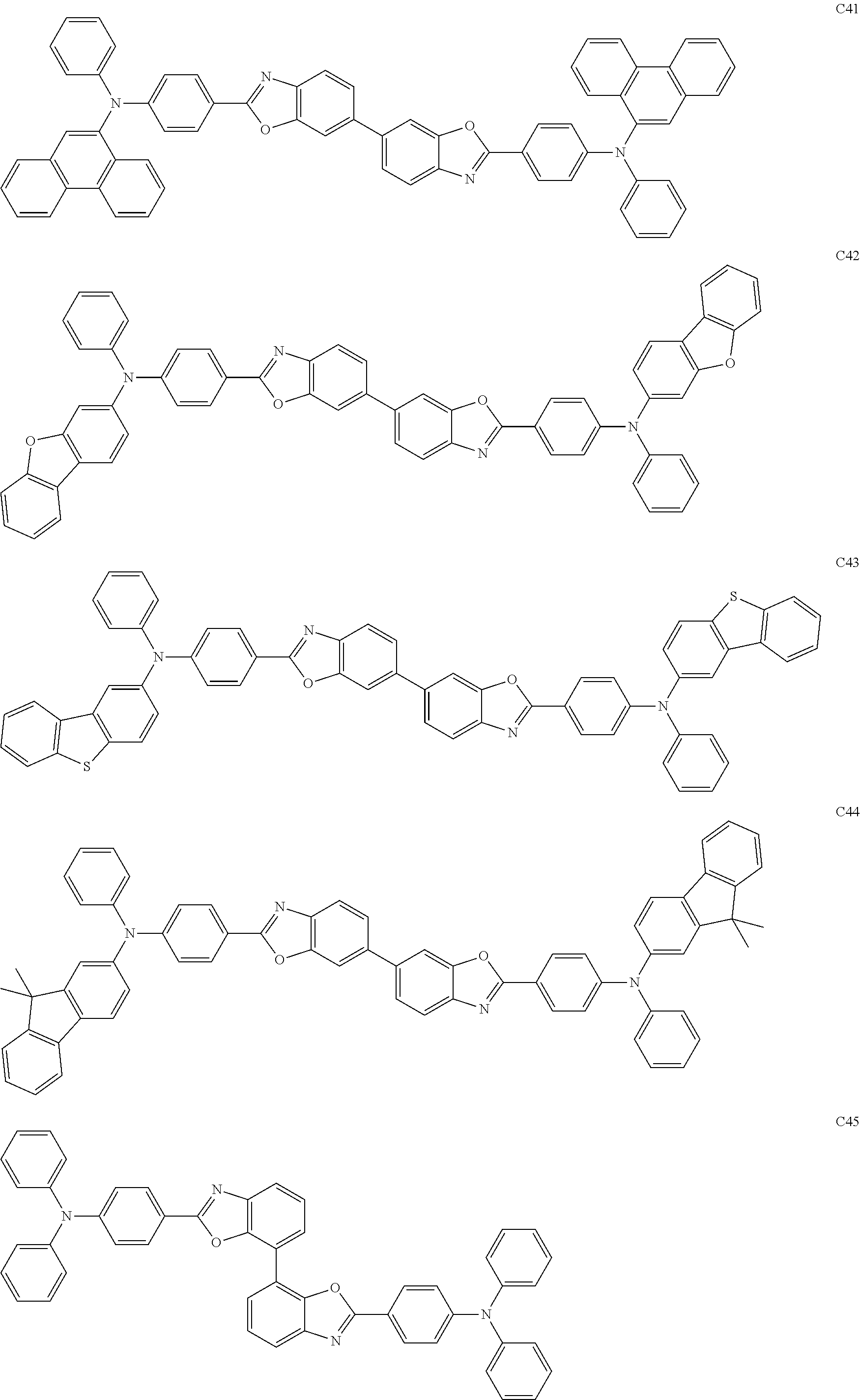

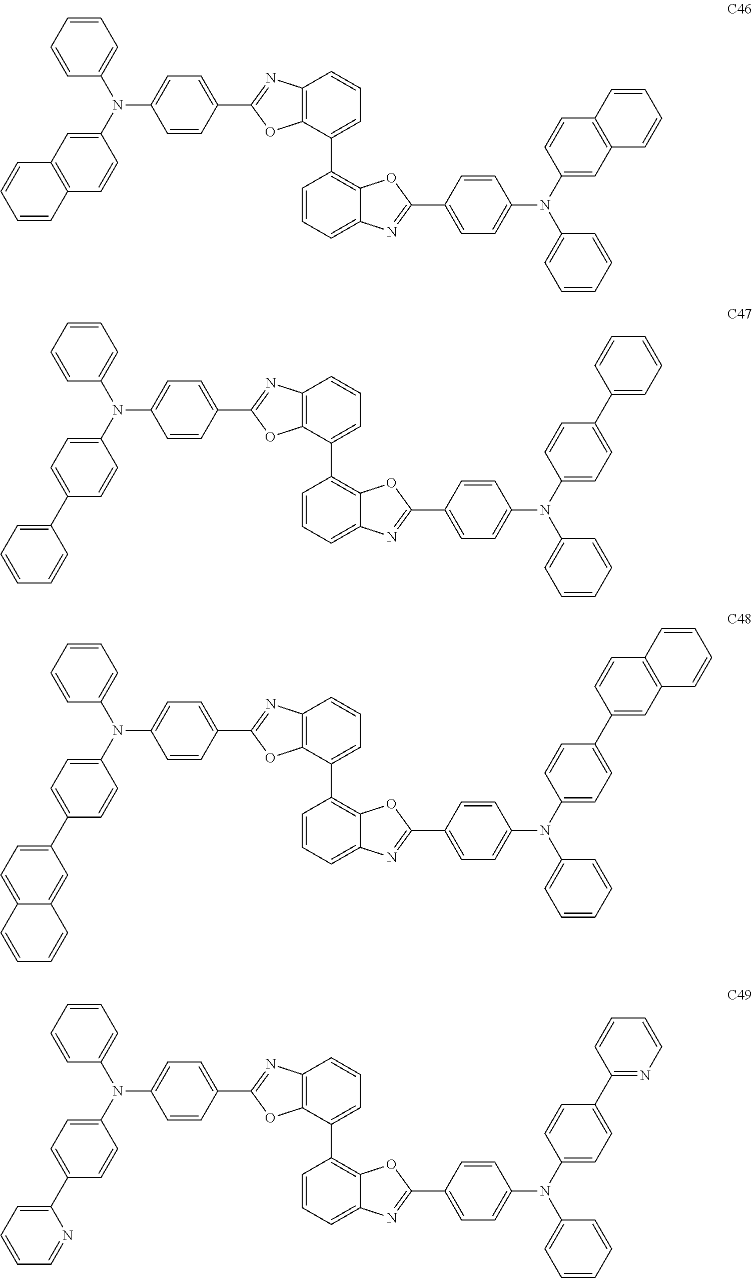

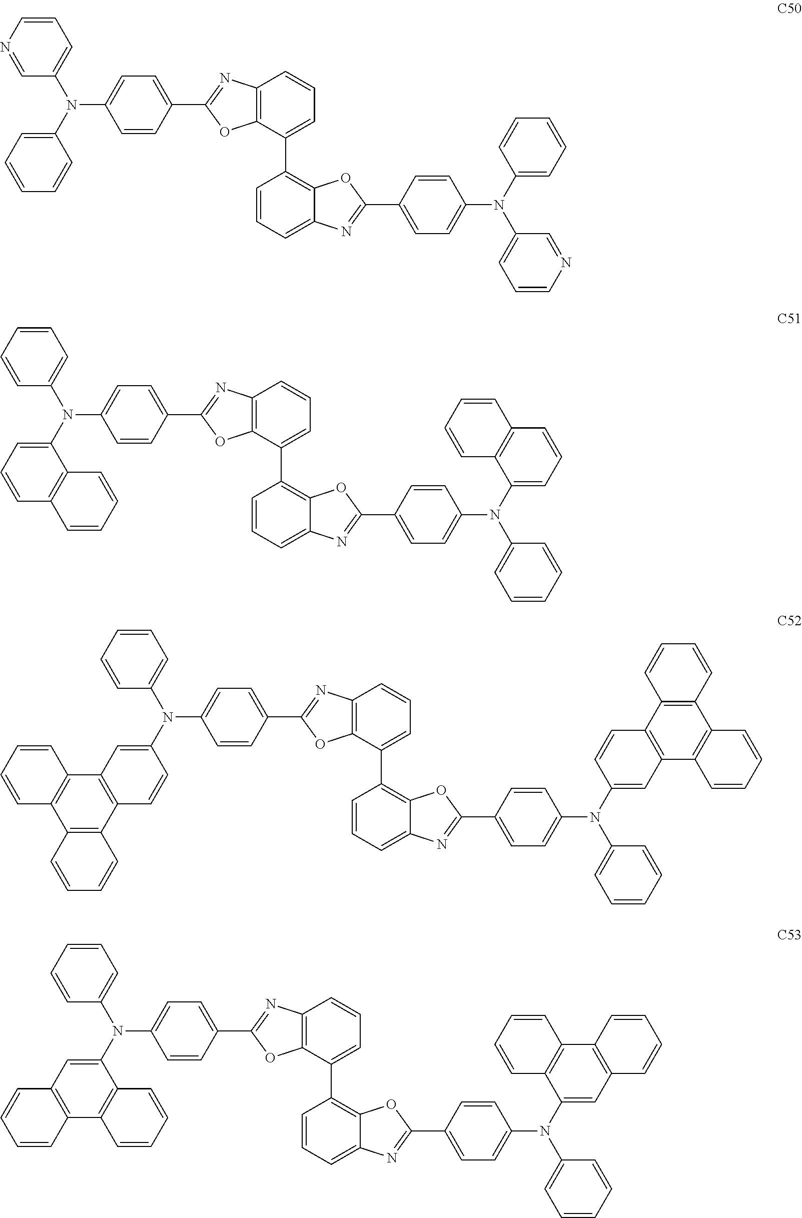 [SMILES: c1ccc(N(c2ccc(-c3nc4cccc(-c5cccc6nc(-c7ccc(N(c8ccccc8)c8cccnc8)cc7)oc56)c4o3)cc2)c2cccnc2)cc1 ; c1ccc(N(c2ccc(-c3nc4cccc(-c5cccc6nc(-c7ccc(N(c8ccccc8)c8cccc9ccccc89)cc7)oc56)c4o3)cc2)c2cccc3ccccc23)cc1 ; c1ccc(N(c2ccc(-c3nc4cccc(-c5cccc6nc(-c7ccc(N(c8ccccc8)c8ccc9c%10ccccc%10c%10ccccc%10c9c8)cc7)oc56)c4o3)cc2)c2ccc3c4ccccc4c4ccccc4c3c2)cc1 ; c1ccc(N(c2ccc(-c3nc4cccc(-c5cccc6nc(-c7ccc(N(c8ccccc8)c8cc9ccccc9c9ccccc89)cc7)oc56)c4o3)cc2)c2cc3ccccc3c3ccccc23)cc1]

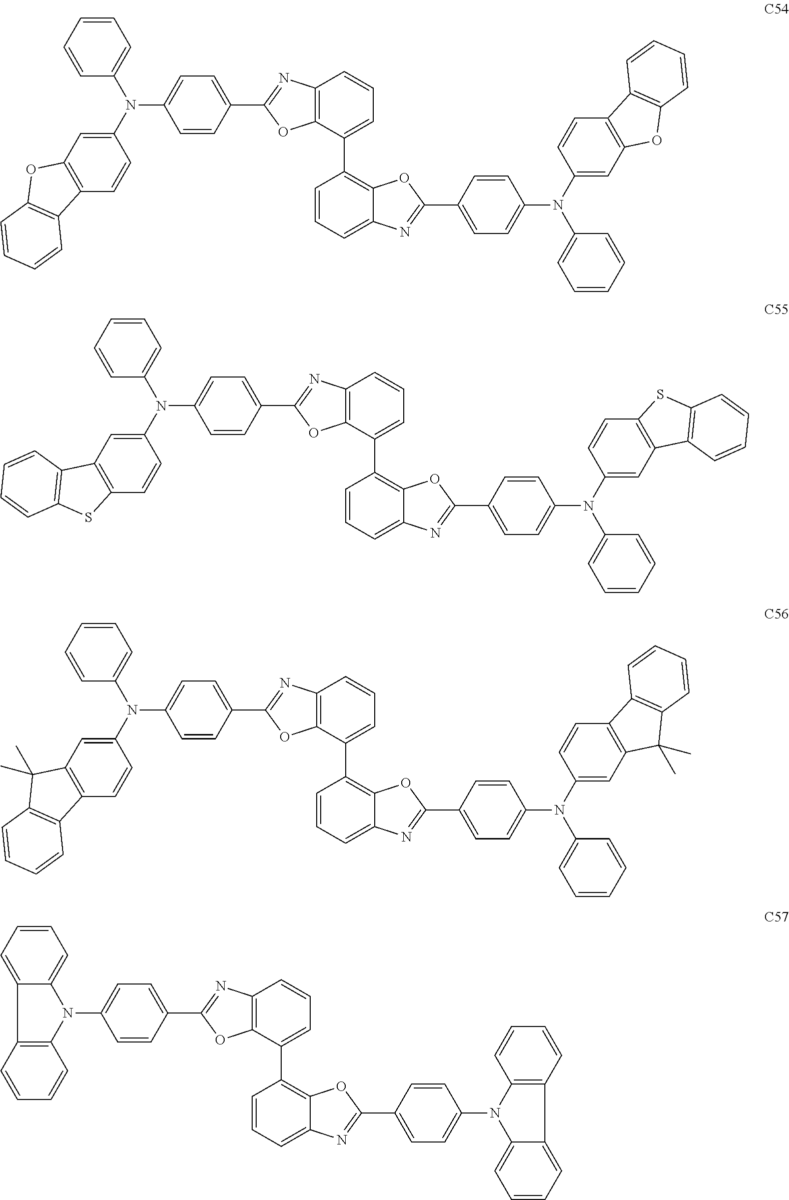

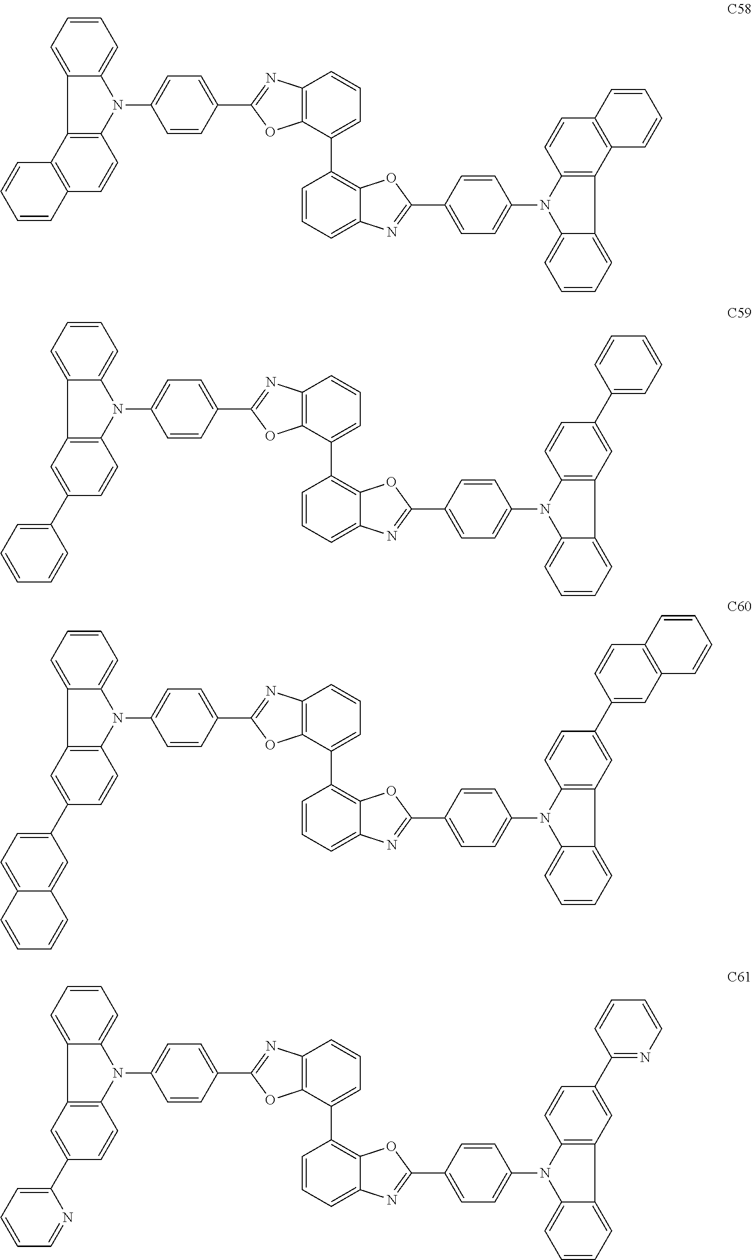

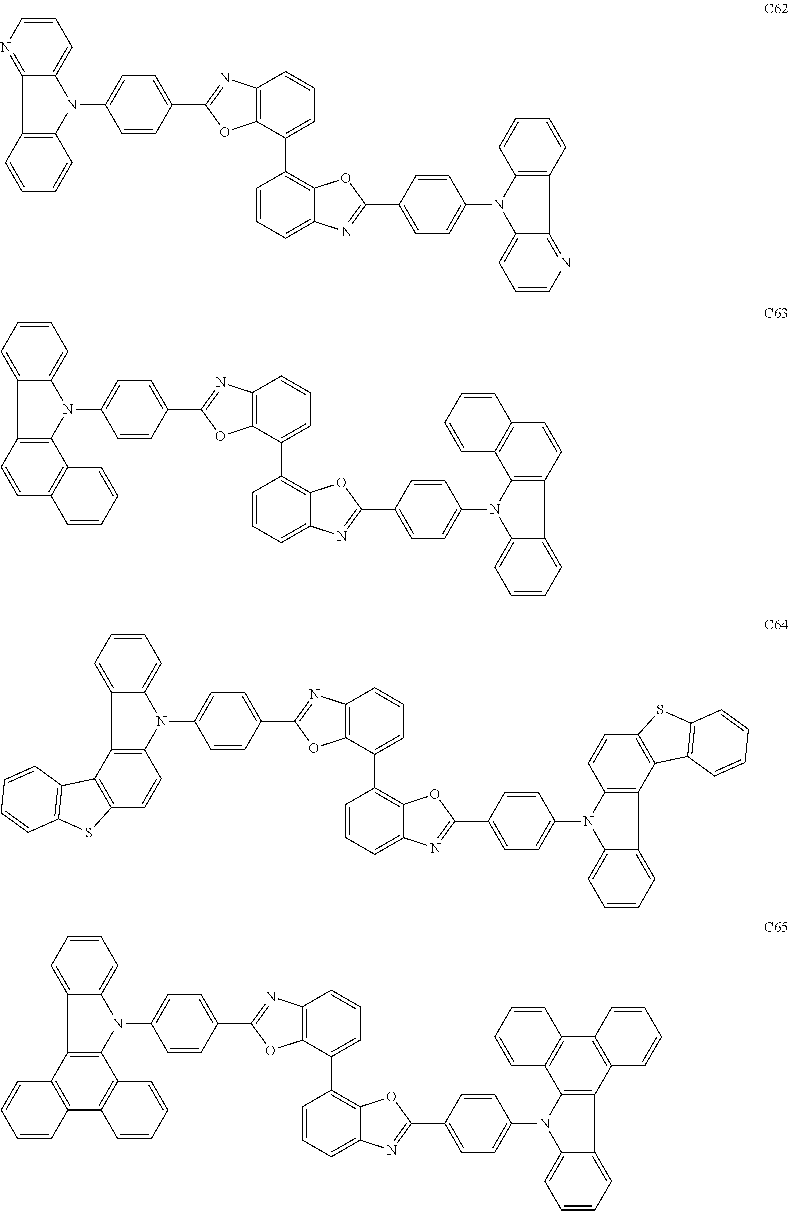 [SMILES: c1cc(-c2cccc3nc(-c4ccc(-n5c6ccccc6c6ncccc65)cc4)oc23)c2oc(-c3ccc(-n4c5ccccc5c5ncccc54)cc3)nc2c1 ; c1ccc2c(c1)ccc1c3ccccc3n(-c3ccc(-c4nc5cccc(-c6cccc7nc(-c8ccc(-n9c%10ccccc%10c%10ccc%11ccccc%11c%109)cc8)oc67)c5o4)cc3)c21 ; c1cc(-c2cccc3nc(-c4ccc(-n5c6ccccc6c6c7c(ccc65)sc5ccccc57)cc4)oc23)c2oc(-c3ccc(-n4c5ccccc5c5c6c(ccc54)sc4ccccc46)cc3)nc2c1 ; c1cc(-c2cccc3nc(-c4ccc(-n5c6ccccc6c6c7ccccc7c7ccccc7c65)cc4)oc23)c2oc(-c3ccc(-n4c5ccccc5c5c6ccccc6c6ccccc6c54)cc3)nc2c1]

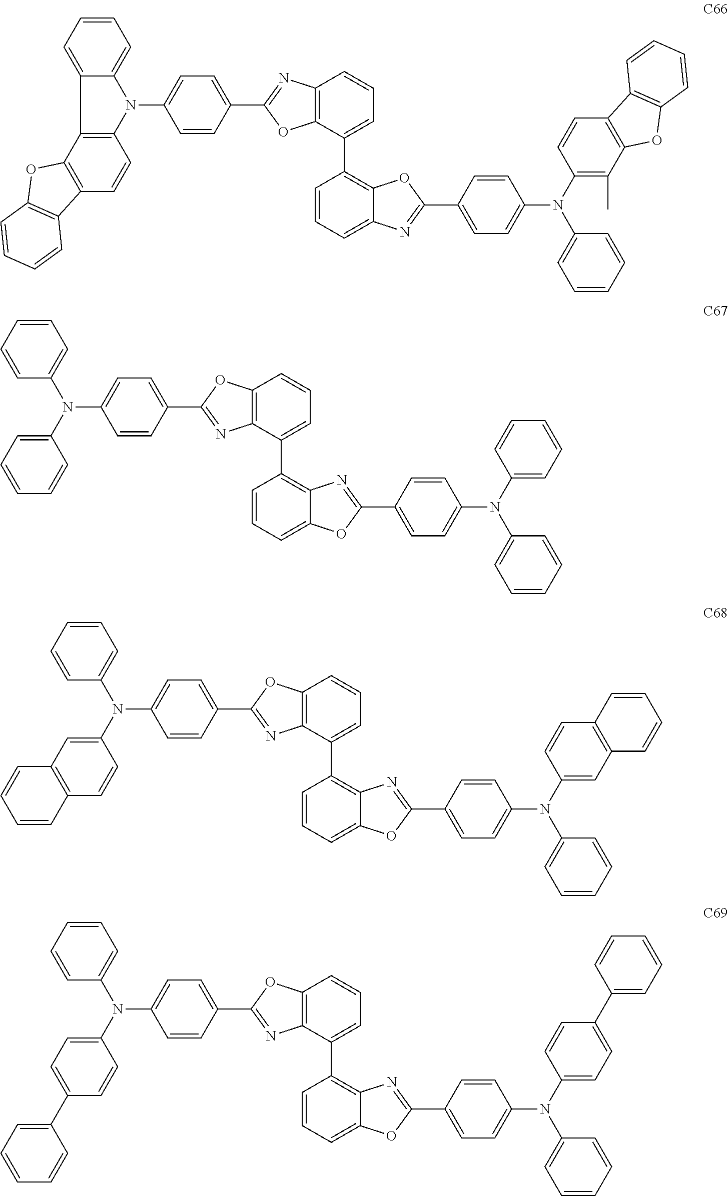

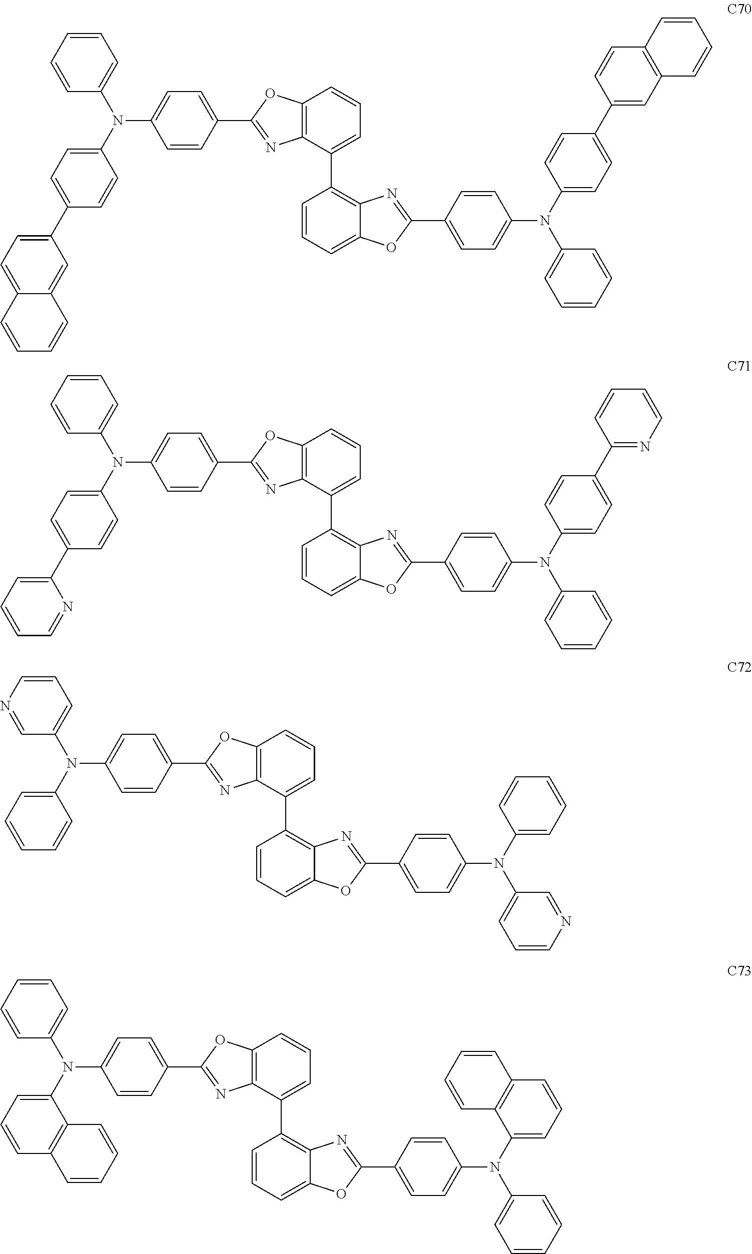

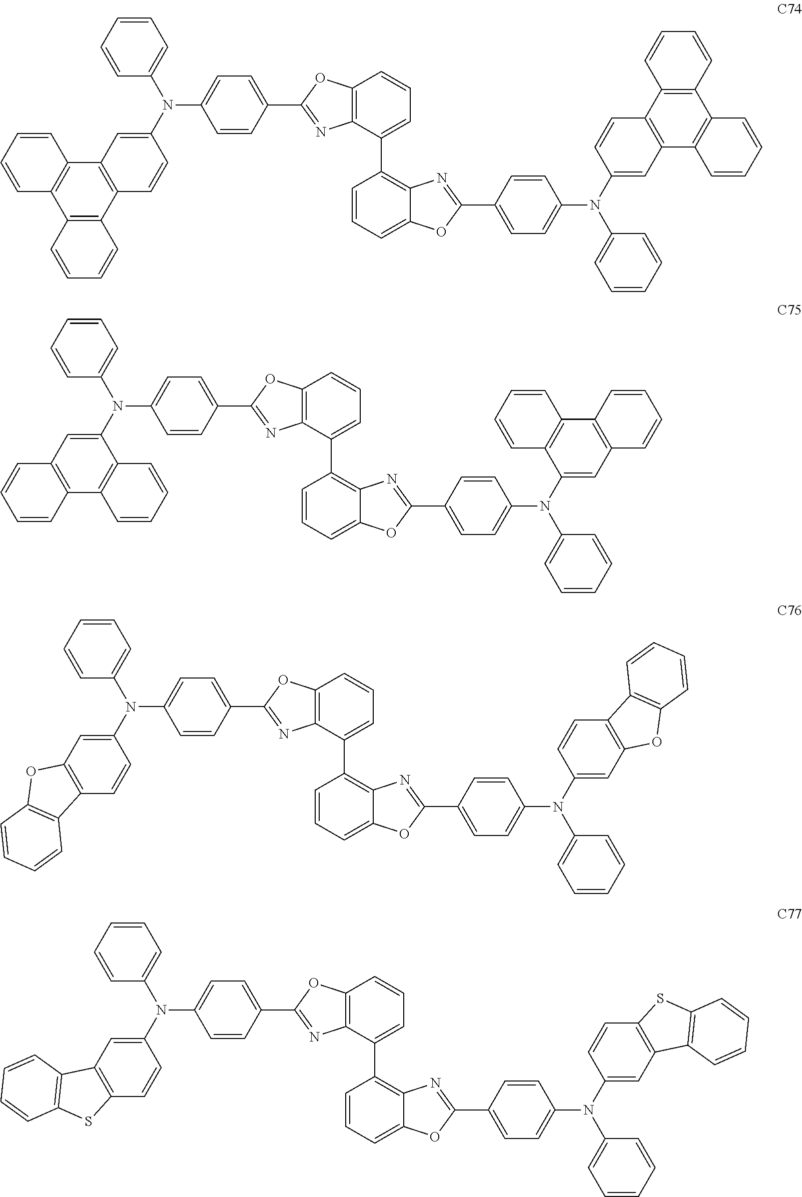

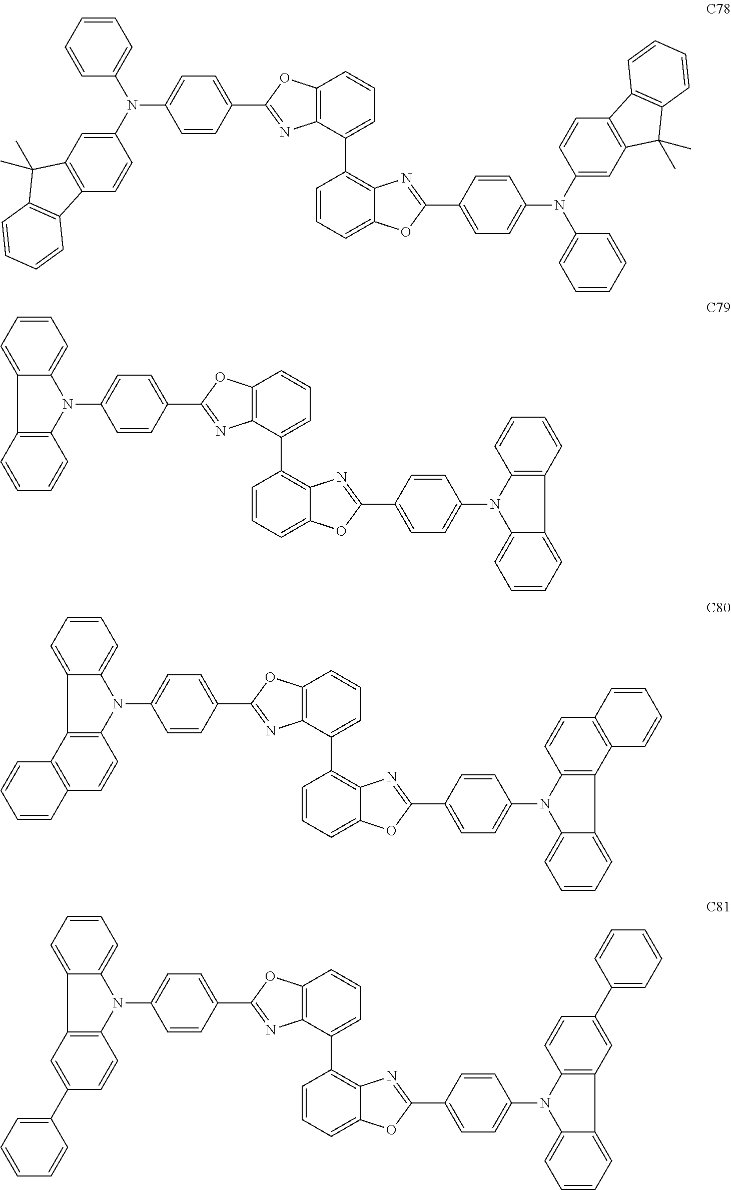

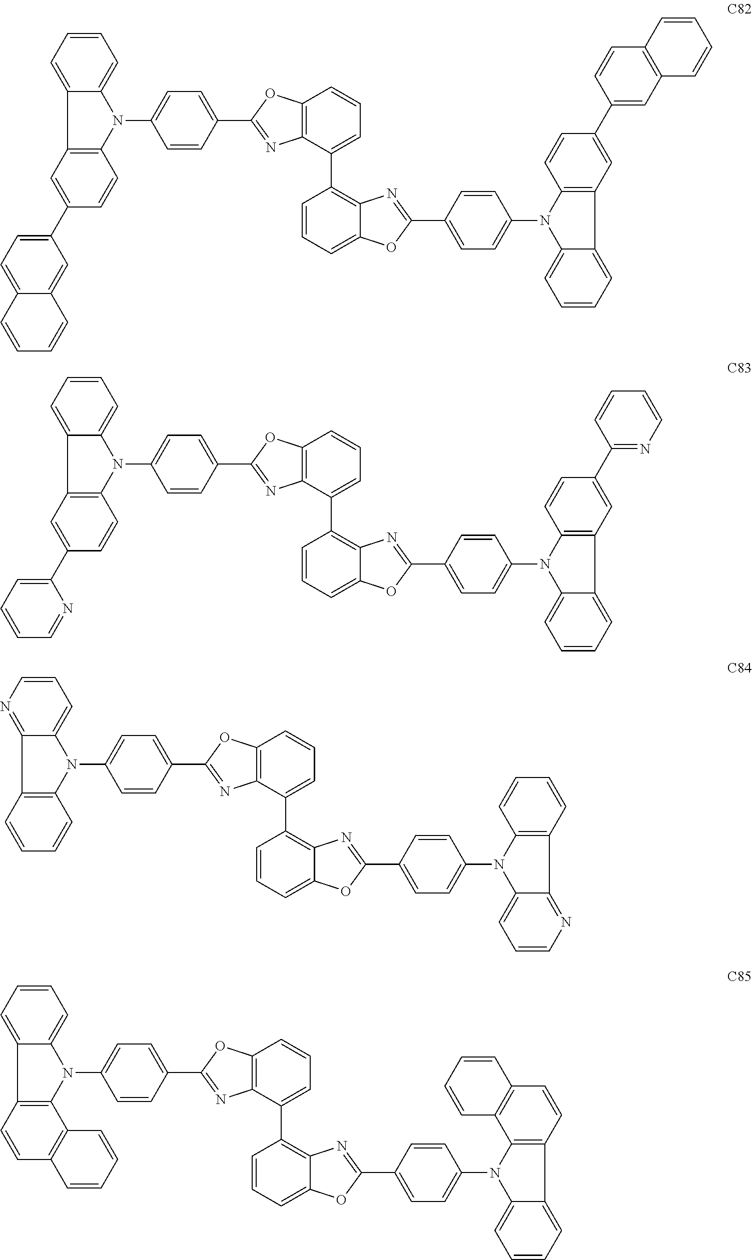

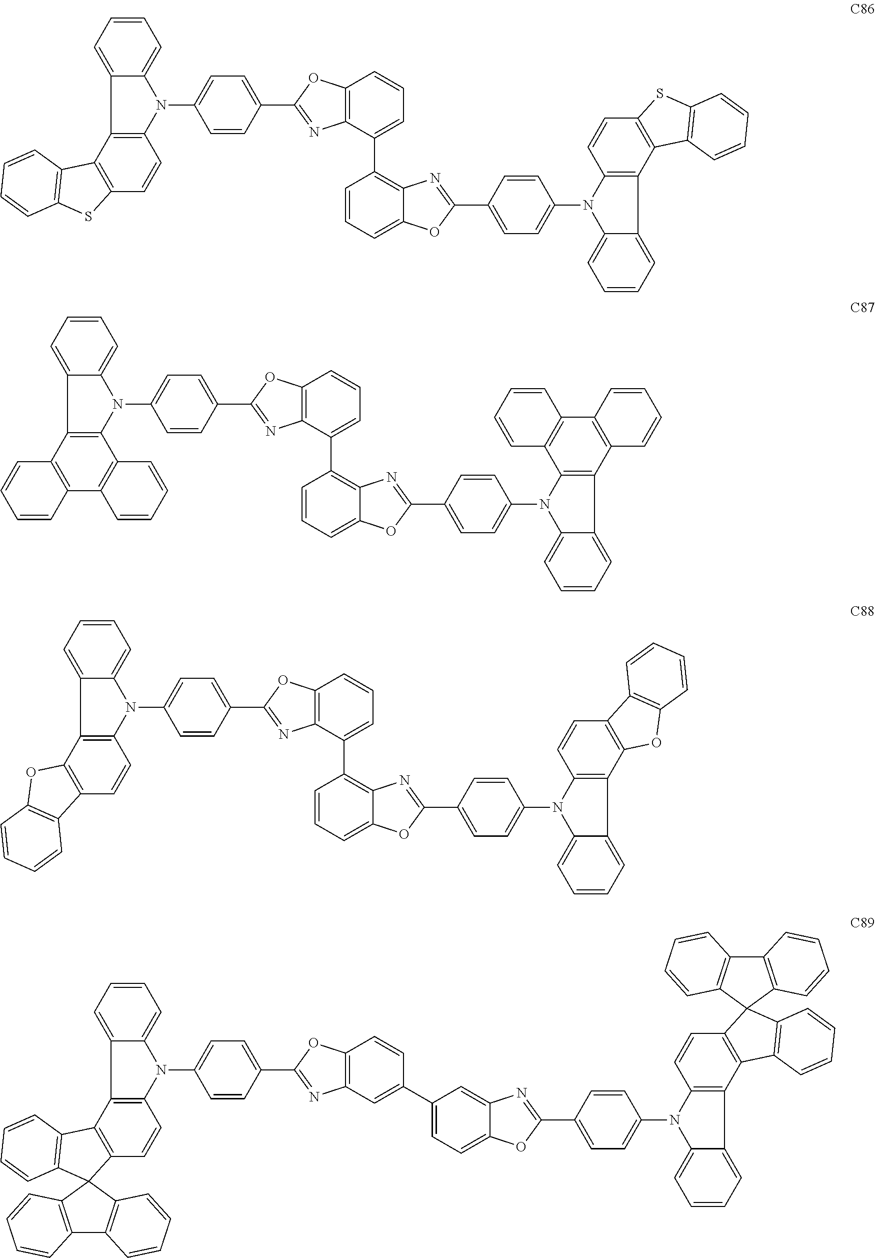

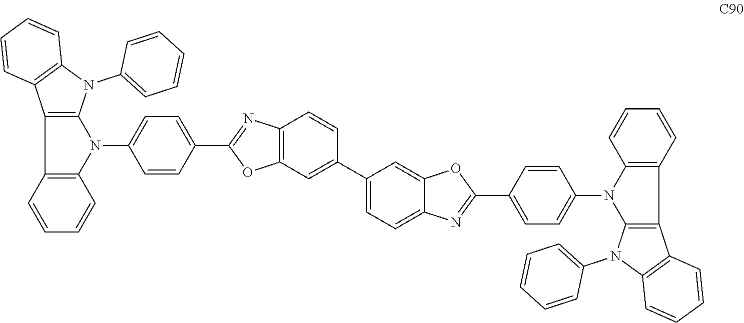 [SMILES: c1ccc(-n2c3ccccc3c3c4ccccc4n(-c4ccc(-c5nc6ccc(-c7ccc8nc(-c9ccc(-n%10c%11ccccc%11c%11c%12ccccc%12n(-c%12ccccc%12)c%11%10)cc9)oc8c7)cc6o5)cc4)c32)cc1]

The compound according to the present disclosure is used for the organic electroluminescent device.

In an embodiment, the compound may be used as a light extraction layer material in the functional layer of the electronic device.

The present disclosure further relates to a mixture. The mixture includes at least one or more compounds for the organic electroluminescent device, and at least another organic functional material. The at least another organic functional material may be selected from a hole injection material (HIM), a hole transfer material (HTM), an electron transfer material (ETM), an electron injection material (EIM), and an electron blocking material (EBM), a hole blocking material (HBM), a light-emitting material (Emitter), a host material (Host), and an organic dye. For example, detailed descriptions of various organic functional materials are provided in patent applications WO2010135519A1, US20090134784A1, and WO 2011110277A1. All contents of these three patent documents are hereby incorporated into this disclosure for reference.

The present disclosure further relates to a composition. The composition includes at least one organic compound or one mixture for the organic electroluminescent device as described above, and at least one organic solvent. The at least one organic solvent is selected from aryl, heteroaryl, ester, aryl ketone, aryl ether, aliphatic ketone, aliphatic ether, an alicyclic compound, an olefin compound, a boric acid ester-based compound or a phosphate ester-based compound, or a mixture of two or more solvents.

In a preferred embodiment, according to the composition of the present disclosure, the at least one organic solvent is selected from an aryl or heteroaryl solvent.

Examples of aryl-based or heteroaryl solvents suitable for the present disclosure include, but not limited to: p-diisopropylbenzene, pentylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, tripentylbenzene, pentyltoluene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylisopropylbenzene, 1-methylnaphthalene, 1,2,4-trichlorobenzene, 4,4-difluorodiphenylmethane, 1,2-dimethoxy-4-(1-propenyl) benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl) pyridine, benzyl benzoate, 1,1-bis (3,4-dimethylphenyl) ethane, 2-isopropyl naphthalene, quinoline, isoquinoline, methyl 2-furancarboxylate, ethyl 2-furancarboxylate, or the like.

Examples of the aryl ketone-based solvent suitable for the present disclosure include, but not limited to: 1-tetralone, 2-tetralone, 2-(phenylepoxy)-tetralone, 6-(methoxy)-tetralone, acetophenone, phenylpropanone, benzophenone, and derivatives thereof, such as 4-methyl acetophenone, 3-methyl acetophenone, 2-methyl acetophenone, 4-methyl phenylpropanone, 3-methyl phenylpropanone, 2-methyl phenylpropanone, or the like.

Examples of aryl ether-based solvents suitable for the invention include, but not limited to: 3-phenoxytoluene, butoxybenzene, p-anisaldehyde dimethyl acetal, tetrahydro-2-phenoxy-2H-pyran, 1,2-dimethoxy-4-(1-propenyl) benzene, 1,4-benzodioxane, 1,3-dipropylbenzene, 2,5-dimethoxytoluene, 4-ethoxyethyl ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-p-propenyl anisole, 1,2-dimethoxybenzene, 1-methoxynaphthalene, diphenyl ether, 2-phenoxymethyl ether, 2-phenoxytetrahydrofuran, ethyl-2-naphthyl ether.

In some preferred embodiments, according to the composition of the present disclosure, the at least one solvent may be selected from: aliphatic ketones, such as 2-nonanone, 3-nonanone, 5-nonanone, 2-decanone, 2,5-hexanedione, 2,6,8-trimethyl-4-nonanone, fenchone, phorone, isophorone, di-n-pentyl ketone, or the like, or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether,or the like.

In other preferred embodiments, according to the composition of the present disclosure, the at least one solvent may be selected from ester-based solvents: octanoic acid alkyl ester, sebacic acid alkyl ester, stearic acid alkyl ester, benzoic acid alkyl ester, phenylacetic acid alkyl ester, cinnamic acid alkyl ester, oxalic acid alkyl ester, maleic acid alkyl ester, alkyl lactone, oleic acid alkyl ester, or the like, especially preferably octyl caprylate, diethyl sebacate, diallyl phthalate, isononyl isononate.

The solvent may be used alone or as a mixture of two or more organic solvents.

In certain preferred embodiments, according to the composition of the present disclosure, the composition includes at least one organic compound, a polymer, or a mixture as described above, and at least one organic solvent. The composition further includes another organic solvent. Another example of the organic solvent includes (but is not limited to): methanol, ethanol, 2-methoxyethanol, dichloromethane, chloroform, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, naphthalene, indene and/or their mixtures.

In some preferred embodiments, the solvent particularly suitable for the present disclosure is a solvent with Hansen solubility parameters within the following range.

δd (dispersion force) ranges from 17.0 to 23.2 MPa½, especially range from 18.5 to 21.0 MPa½;

δd (polarity force) ranges from 0.2 to 12.5 MPa½, especially ranges from 2.0 to 6.0 MPa ½; and

δh (hydrogen bonding force) ranges from 0.9˜14.2 MPa½, especially ranges from 2.0 to 6.0 MPa½.

For the composition of the present disclosure, the boiling point of an organic solvent needs to be considered when selecting the organic solvent. In the present disclosure, the boiling point of the organic solvent is greater than or equal to 150° C., preferably greater than or equal to 180° C., further preferably greater than or equal to 200° C., more preferably greater than or equal to 250° C., and most preferably greater than or equal to 275° C. or greater than or equal to 300° C. The boiling points within these ranges are beneficial for preventing blockage of nozzle of inkjet printing heads. The organic solvent may be evaporated from the solvent system to form a thin film containing functional materials.

In a preferred embodiment, the composition according to the present disclosure is a solution.

In another preferred embodiment, the composition according to the present disclosure is a suspension.

The composition in some embodiments of the present disclosure may include 0.01 wt % to 10 wt % of the compound or mixture according to the present disclosure, preferably 0.1 wt % to 15 wt %, more preferably 0.2 wt % to 5 wt %, and most preferably 0.25 wt % to 3 wt %.

The present disclosure further relates to the use of the composition as a coating or printing ink in the preparation of the organic electronic device, particularly preferably by printing or coating preparation methods.

Suitable printing or coating methods include, but not limited to, inkjet printing, spray printing (Nozzle Printing), typography, screen printing, dip coating, rotary coating, scraper coating, roller printing, torsion roller printing, lithography, flexographic printing, rotary printing, spraying, brushing or transfer printing, slit-type extrusion coating, or the like. The preferred is gravure printing, spray printing and inkjet printing. The solution or suspension may include one or more components such as surface activation compounds, lubricants, wetting agents, dispersants, hydrophobic agents, adhesives, or the like, for regulating viscosity, film forming performance, improving adhesion, or the like. Specific printing method may have relevant requirements for the solution, such as solvent, concentration, viscosity, or the like.

The present disclosure further relates to an organic electroluminescent device. The organic electroluminescent device includes two electrodes, one or more organic functional layers disposed between the two electrodes, and a light extraction layer disposed on a surface of one electrode away from the organic functional layer. In particular, a material of the light extraction layer includes the compound as represented by formula (1).

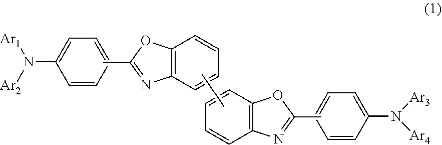

in which:

Ar₁ to Ar₄ are independently selected from the group consisting of a substituted or unsubstituted aromatic group having 6 to 40 ring atoms, and a substituted or unsubstituted heteroaromatic group having 5 to 40 ring atoms.

Ar₁ and Ar₂ form a ring or do not form a ring. Ar₃ and Ar₄ form a ring or do not form a ring.

Further explanation of the formula (1) is as described earlier.

According to the organic electroluminescent device described in the present disclosure, the material of the light extraction layer requires a higher glass transition temperature to enhance the thermal stability of the material of the light extraction layer. In some preferred embodiments, the glass transition temperature T_{g}≥100° C. In a preferred embodiment, T_{g}≥120° C. In a further preferred embodiment, T_{g}≥140° C., in a more preferred embodiment, T_{g}≥160° C. In a most preferred embodiment, T_{g}≥180° C.

In certain embodiments, according to the organic electroluminescent device described in the present disclosure, the refractive index of the material of the light extraction layer at a wavelength of 630 nm is greater than 1.7, preferably, greater than 1.78, and more preferably, greater than 1.83.

According to the organic electroluminescent device described in the present disclosure, the material of the light extraction layer requires to have a smaller extinction coefficient, and the extinction coefficient is less than 0.1 at a wavelength of 430 nm, preferably, less than 0.003, and more preferably, less than 0.001. The material of the light extraction layer has a high transmittance to visible light and reduces the impact on light output efficiency of the device.

In certain preferred embodiments, according to the organic electroluminescent device of the present disclosure, the light extraction layer has a larger extinction coefficient in a wavelength range of less than or equal to 400 nm. Preferably, the extinction coefficient is greater than or equal to 0.3 at a wavelength of 350 nm. Further preferably, the extinction coefficient is greater than or equal to 0.5. More preferably, the extinction coefficient is greater than or equal to 0.7. Most preferably, the extinction coefficient is greater than or equal to 1.0.

In an embodiment, the organic electroluminescent device according to the present disclosure has the light extraction layer located on a surface of a cathode surface.

In an embodiment, the organic electroluminescent device according to the present disclosure includes one or more organic functional layers. The organic functional layer is selected from one or more layers of an electron injection layer, an electron transport layer, a hole injection layer, a hole transport layer, and a light-emitting layer. At least the light-emitting layer is included.

In certain preferred embodiments, according to the organic electroluminescent device of the present disclosure, a luminescent material of the light-emitting layer is selected from a singlet emitter, a triplet emitter, or a thermally activated delayed fluorescence (TADF) material.

In some preferred embodiments, according to the organic electroluminescent device of the present disclosure, the organic functional layer is selected from the hole transport layer, the light-emitting layer, and the electron transport layer.

In certain preferred embodiments, according to the organic electroluminescent device of the present disclosure, the organic functional layer is selected from the hole injection layer, the hole transport layer, the light-emitting layer, the electron transport layer, and the electron injection layer.

For the details of the singlet emitter, the triplet emitter, and a TADF material, please refer to, for example, WO2017/092619A.

Furthermore, according to the organic electroluminescent device of the present disclosure, the organic functional layer includes the light-emitting layer. Furthermore, the luminescent material of the light-emitting layer is selected from the triplet emitter.

In an embodiment, the triplet emitter has the following general formula.

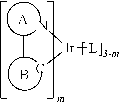

In which, m is selected from 1, 2, or 3.

Ring A is selected from a substituted or unsubstituted N-containing heteroaromatic group having 5 to 30 ring atoms.

Ring B is selected from a substituted or unsubstituted aromatic or heteroaromatic group having 6 to 30 ring atoms.

L is a monovalent anionic organic ligand.

In an embodiment, ring A is selected from the group consisting of the following groups.

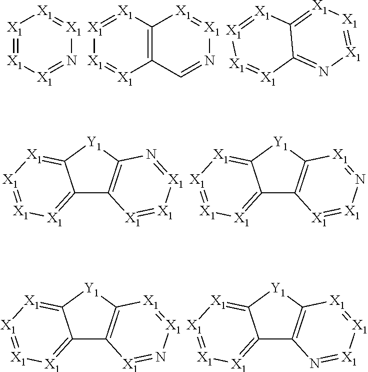

In which, N atom is the atom coordinated with Ir.

In an embodiment, ring B is selected from the group consisting of the following groups.

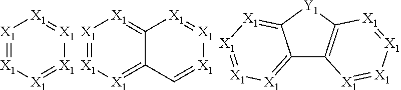

In which:

At each occurrence, X₁ is independently selected from CR₆ or N;

At each occurrence, Y₁ is independently selected from the group consisting of CR₇R₈, NR₇, O, S, S—O, SO₂, PR₇, BR₇, and SiR₇R₈.

At each occurrence, R₆, R₇, and R₈ are independently selected from the group consisting of —H, -D, a linear alkyl group having 1-20 carbon atoms, a branched or cyclic alkyl group having 3 to 20 carbon atoms, a cyano group, a nitro group, —CF₃, —OCF₃, —Cl, —Br, —F, a substituted or unsubstituted aromatic group having 6 to 30 ring atoms, a substituted or unsubstituted heteroaromatic group having 5 to 30 ring atoms, or any combination of the groups.

Furthermore, the triplet emitter is selected from the group consisting of the following general formulae.

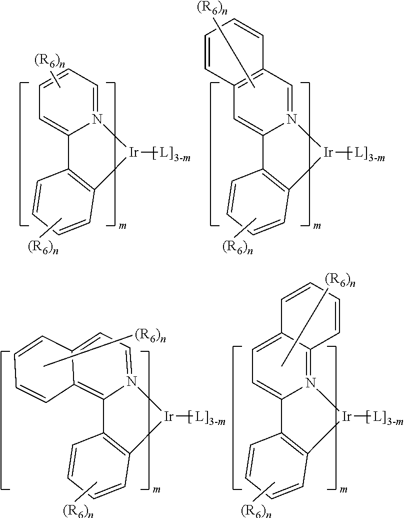

Furthermore, at each occurrence, R₆ is independently selected from the group consisting of —H, -D, a linear alkyl group having 1 to 10 carbon atoms, a branched or cyclic alkyl group having 3 to 10 carbon atoms, a cyano group, a nitro group, —CF₃, —OCF₃, —C₁, —Br, —F, an aromatic group having 6 to 20 ring atoms, a heteroaromatic group having 5 to 20 ring atoms, or any combination of the groups.

At each occurrence, n is independently selected from integers of 0 to 5.

Furthermore, the triplet emitter is selected from the group consisting of the following general formula.

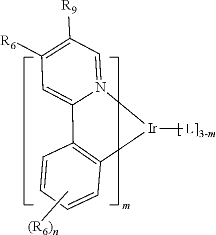

In which, at each occurrence, R₉ is independently selected from the group consisting of —H, -D, a linear alkyl group having 1 to 10 carbon atoms, a branched or cyclic alkyl group having 3 to 10 carbon atoms, a cyano group, a nitro group, —CF₃, —OCF₃, —Cl, —Br, —F, an aromatic group having 6 to 20 ring atoms, a heteroaromatic group having 5 to 20 ring atoms, and any combination of the groups; furthermore, at each occurrence, R₉ is independently selected from the group consisting of phenyl, methyl, and deuterated methyl.

The triplet emitter according to the present disclosure is selected from the group consisting of the following structures.

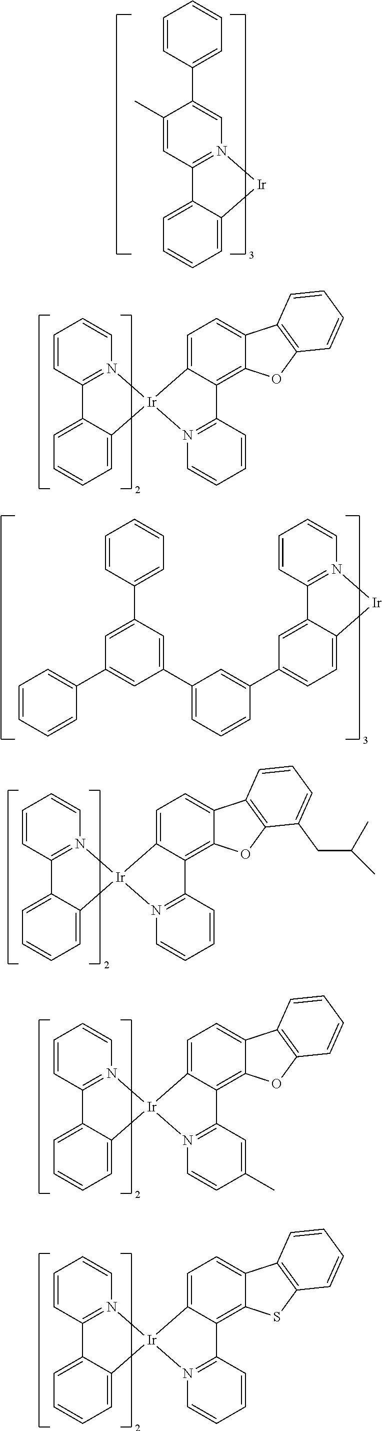

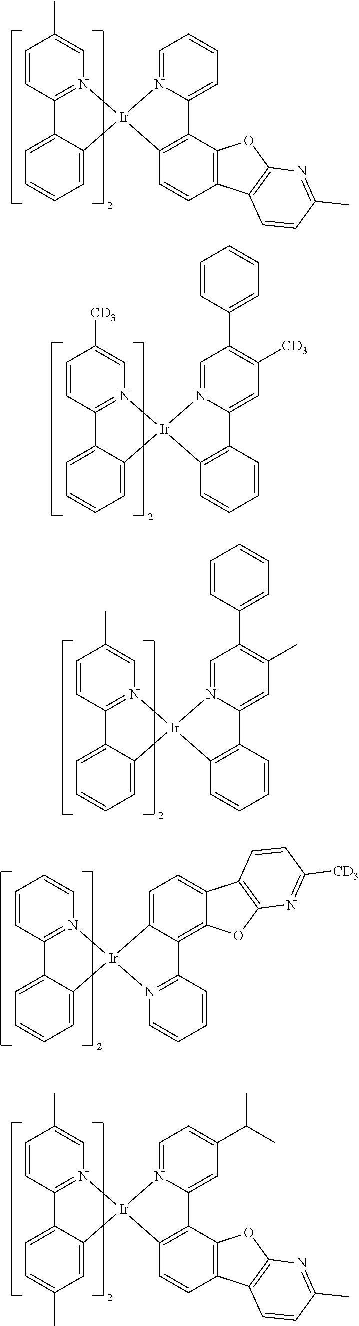

According to the present disclosure, the organic electronic device may be selected from, but are not limited to, an organic light-emitting diode (OLED), an organic photovoltaic cell, an organic light-emitting cell, an organic field-effect transistor, an organic light-emitting field-effect transistor, an organic laser, an organic spintronic device, an organic sensor, an organic plasmon emitting diodes, or the like, especially preferably OLED.

Below is a description of the device structure of the organic light-emitting diodes, including the cathode, the anode, and the light extraction layer, but not limited to these.

The anode may include a conductive metal, metal oxide, or conductive polymer. The anodes may easily inject holes into the hole injection layer (HIL), hole transport layer (HTL), or light-emitting layer. In an embodiment, the absolute difference between the work function of the anode, and the highest occupied molecular orbital (HOMO) energy level or valence band energy level of the luminescent body of the light-emitting layer or p-type semiconductor materials used as HIL, HTL, or electron barrier layer (EBL) is less than 0.5 eV, preferably less than 0.3 eV, and more preferably less than 0.2 eV. Examples of the anode material includes, but not limited to, Al, Cu, Au, Ag, Mg, Fe, Co, Ni, Mn, Pd, Pt, ITO, aluminum doped zinc oxide (AZO), or the like. Other suitable anode materials are known and may be easily chosen for use by those skilled in the art. The anode material may be deposited using any suitable method, for example, a suitable physical vapor deposition method, including radio frequency magnetron sputtering, vacuum thermal evaporation, electron beam (e-beam), or the like. In some embodiments, the anode is patterned and structured. A patterned ITO conductive substrate may be purchased on the market and may be used to prepare the device according to the present disclosure.

The cathode may include a conductive metal or metal oxide. The cathode may easily inject electrons into EIL or ETL, or directly into the light-emitting layer. In an embodiment, the absolute difference between the work function of the cathode, and the lowest unoccupied molecular orbital (LUMO) energy level or conduction band energy level of the n-type semiconductor material in the emitting layer or as an electron injection layer (EIL), electron transfer layer (ETL), or hole blocking layer (HBL) is less than 0.5 eV, preferably less than 0.3 eV, and preferably less than 0.2 eV. In principle, all materials that may be used as the cathode for OLED may be used as the cathode material for the device of the present disclosure. Examples of the cathode material include but not limited to Al, Au, Ag, Ca, Ba, Mg, LiF/Al, MgAg alloys, BaF₂/Al, Cu, Fe, Co, Ni, Mn, Pd, Pt, ITO, or the like. The cathode material may be deposited using any suitable technique, such as a suitable physical vapor deposition method, including radio frequency magnetron sputtering, vacuum thermal evaporation, electron beam (e-beam), or the like.

The material of the light extraction layer needs to have a suitable energy level structure, with strong absorption in areas with wavelengths less than 400 nm, and weak or near zero absorption in visible light with wavelengths greater than 400 nm, to avoid damage to the internal materials of the device caused by high-energy light irradiation in the subsequent process. Moreover, the material of the light extraction layer has a high refractive index, which may effectively export visible light emission and improve the luminous efficiency of the organic electronic light-emitting device. When the reflectivity of the interface between the material of the light extraction layer and an adjacent electrode is high, the influence of light interference is significant. Therefore, the refractive index of the material that constitutes the light extraction layer is preferably greater than the refractive index of the adjacent electrode.

In some embodiments, in the organic electroluminescent device according to the present disclosure, particularly in the organic light-emitting diode, the light extraction layer is located on the surface of the cathode.

In some more preferred embodiments, according to the organic electroluminescent device of the present disclosure, a thickness of an organic compound of the light extraction layer ranges from generally 10 nm to 200 nm, preferably 20 nm to 150 nm, more preferably 30 nm to 100 nm, and most preferably 40 nm to 90 nm.

The present disclosure further relates to a use of the electroluminescent device according to the present disclosure in various electronic devices, including, but not limited to, a display device, a lighting device, a light source, a sensor, or the like.

The present disclosure will be explained in conjunction with preferred embodiments, but the present disclosure is not limited to the following embodiments. It should be understood that the claims summarize the scope of the present disclosure. Under the guidance of the present disclosure concept, those skilled in the art should be aware that certain changes made to each embodiment of the present disclosure will be covered by the spirit and scope of the claims of the present disclosure.

### EXAMPLES

### Synthesis of Compound C1

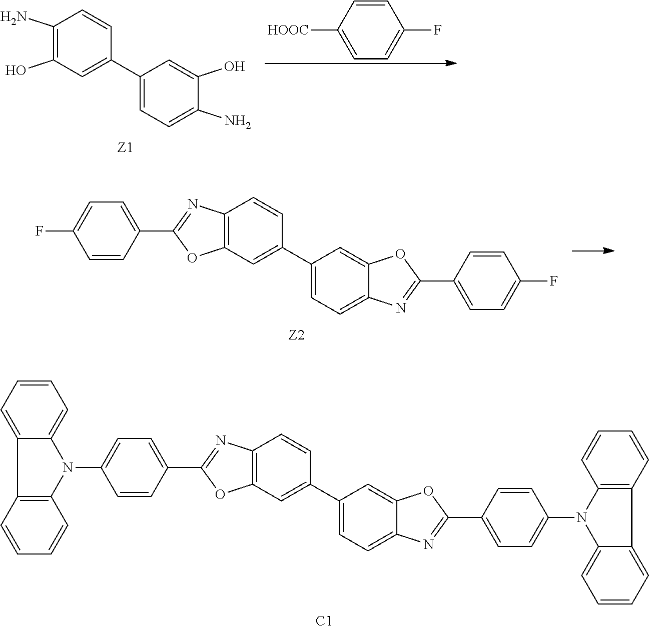 [SMILES: Nc1ccc(-c2ccc(N)c(O)c2)cc1O ; O=C(O)c1ccc(F)cc1 ; Fc1ccc(-c2nc3ccc(-c4ccc5nc(-c6ccc(F)cc6)oc5c4)cc3o2)cc1 ; c1ccc2c(c1)c1ccccc1n2-c1ccc(-c2nc3ccc(-c4ccc5nc(-c6ccc(-n7c8ccccc8c8ccccc87)cc6)oc5c4)cc3o2)cc1 ; "C1"]

Z1 (4.32 g, 20 mmol) and p-fluorobenzoic acid (5.88 g, 42 mmol) were added to polyphosphate acid, gradually heated to 140° C., and stirred. When the isothermal titration calorimetry (TLC) point plate reactant disappeared, the heat source was removed. After the reaction system was cooled, NaOH aqueous solution was added at the presence of ice water bath. The intermediate Z2 was obtained by vacuum filtration with a total of 6.95 g and a yield of 82%.

Z2 (6.36 g, 15 mmol), carbazole (5.34 g, 32 mmol), and cesium carbonate were added to DMF solution. to obtain a mixture. The mixture was gradually heated to 140° C. while stirring. When the TLC point plate reactant disappeared, the heat source was removed. After the reaction system was cooled, water was added. The crude product was obtained by vacuum filtration. A total of 8.1 g of C1 was obtained by washing with toluene, and the yield was 75%. MS: 718 [M⁺].

### Synthesis of Compound C2

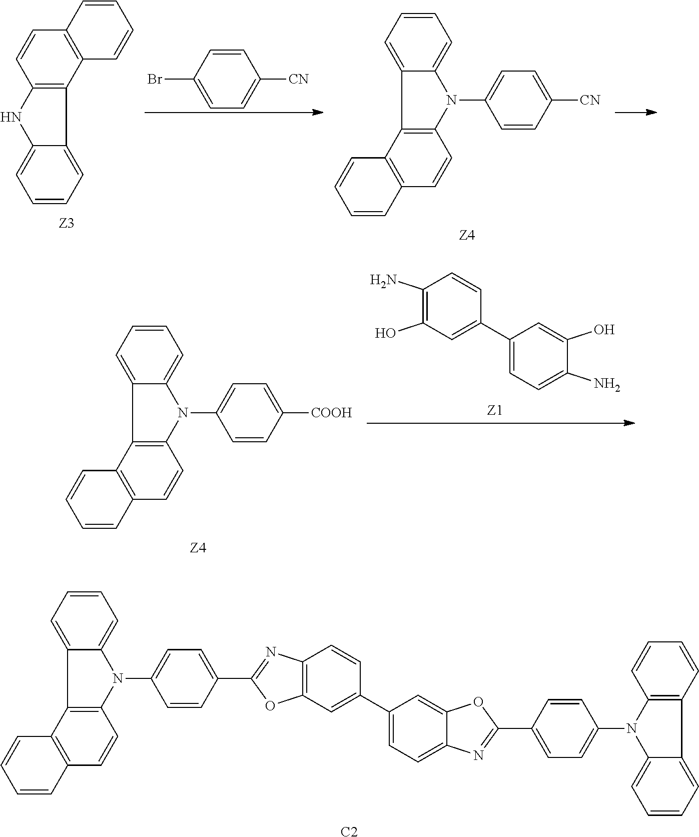

Compound Z3 (6.51 g, 30 mmol) and p-bromobenzonitrile (5,43 g, 30 mmol) were dissolved in anhydrous toluene, to obtain a mixture. Sodium tert-butoxide (3.45 g, 36 mmol) and tris (benzylidene) acetone dipalladium (0.82 g, 0.9 mmol) were added into the mixture. After replacing nitrogen for three times, tri-tert-butylphosphine (0.9 mmol) was added into the mixture. The mixture was gradually heated to 80° C. while stirring for 12 hours, and then the heat source was removed. After the reaction system was cooled, deionized water was added to separate the organic layer, and the organic layer was extracted with ethyl acetate for three times, concentrated under reduced pressure, and purified on silica gel column chromatography to obtain 8.11 g of product Z4 with a yield of 85%.

Z4 (7.95 g, 25 mmol) and NaOH were added to ethanol aqueous solution to obtain a mixture, and the mixture was heated and refluxed. After the reaction was completed, the reacted solution was filtered, acidified with hydrochloric acid, and then extracted with ethyl acetate, washed with water, dried with sodium sulfate and concentrated to obtain a total of 6.15 g of intermediate Z5 with a yield of 73%.

Z1 (1.73 g, 8 mmol) and Z5 (6.07 g, 18 mmol) were added to polyphosphoric acid to obtain a mixture. The mixture was gradually heated to 140° C., while stirring. When the TLC point plate reactant disappeared, the heat source was removed. After the reaction system was cooled, NaOH aqueous solution was added at the presence of ice water bath. The intermediate Z2 was obtained by vacuum filtration with a total of 4.71 g and a yield of 72%. MS: 818 [M⁺].

### Synthesis of Compound C3

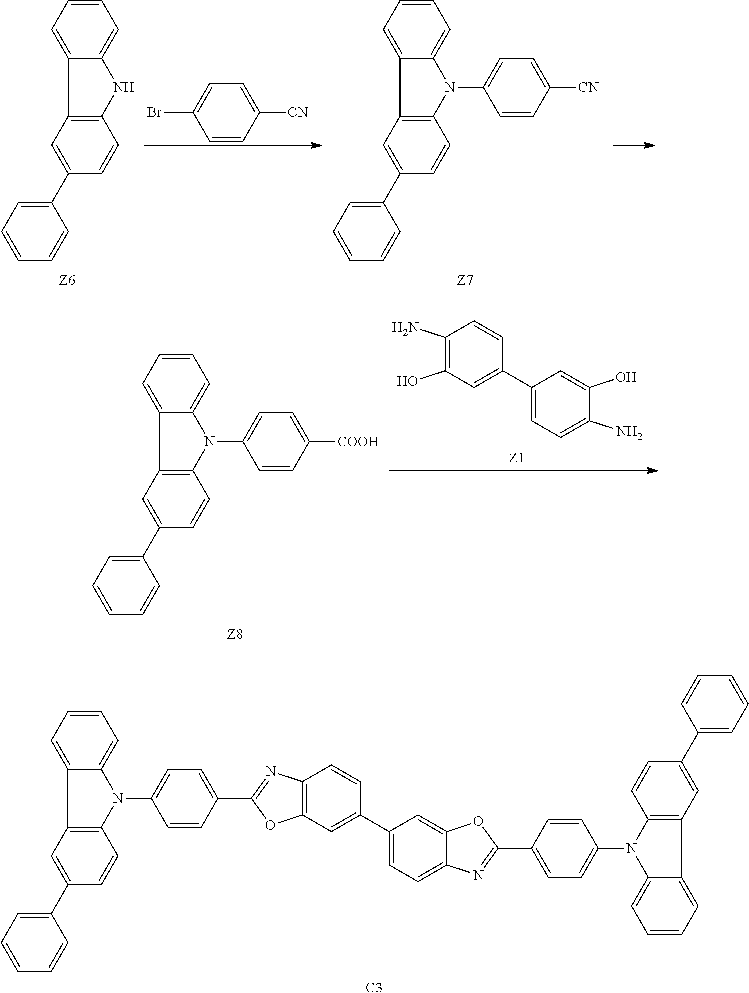

Compound Z6 (7.29 g, 30 mmol) and p-bromobenzonitrile (5.43 g, 30 mmol) were dissolved in anhydrous toluene to obtain a mixture. Sodium tert-butoxide (3.45 g, 36 mmol) and tris (benzylidene) acetone dipalladium (0.82 g, 0.9 mmol) were added into the mixture. After purging the reaction vessel by nitrogen for three times, tri-tert-butylphosphine (0.9 mmol) was added into the mixture. The mixture was gradually heated to 80° C. while stirring 12 hours, and then the heat source was removed. After the reaction system was cooled, deionized water was added to separate the organic layer. The organic layer was extracted with ethyl acetate for three times, concentrated under reduced pressure, and purified on silica gel column chromatography to obtain 7.84 g of product Z7 with a yield of 76%.

Z7 (6.88 g, 20 mmol) and NaOH were added to ethanol aqueous solution to obtained a mixture. The mixture was heated and refluxed. After the reaction was completed, the reacted solution was filtered, acidified with hydrochloric acid, and then extracted with ethyl acetate, washed with water, dried with sodium sulfate and concentrated to obtain intermediate Z8, a total of 4.86 g, with a yield of 67%.

Z1 (1.08 g, 5 mmol) and Z8 (4 g, 11 mmol) were added to polyphosphoric acid to obtain a mixture. The mixture was gradually heated to 140° C., while stirring. When the TLC point plate reactant disappeared, the heat source was removed. After the reaction system was cooled, NaOH aqueous solution was added at the presence of ice water bath. The intermediate C3 was obtained by vacuum filtration with a total of 3.09 g and a yield of 71%. MS: 870 [M⁺].

### Synthesis of Compound C13

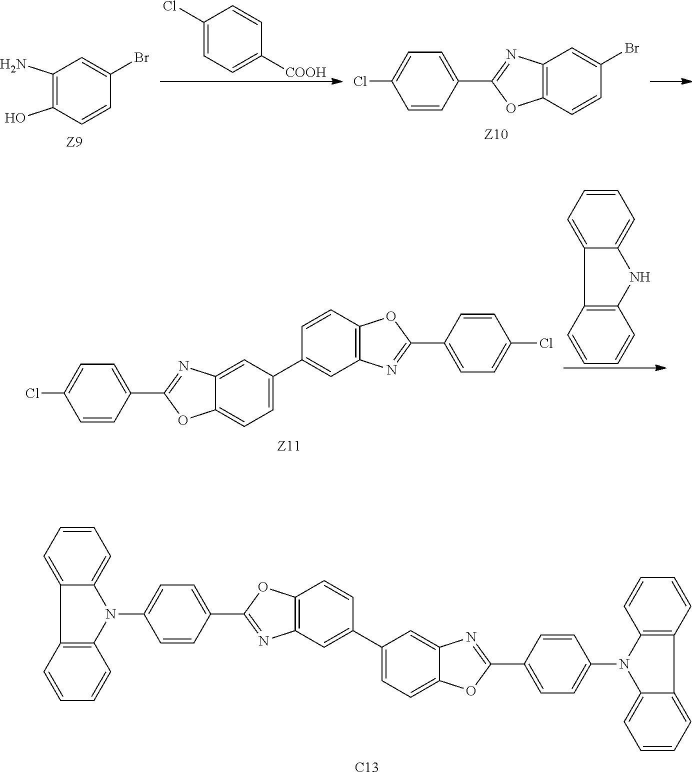

Z9 (3.74 g, 20 mmol) and p-chlorobenzoic acid (3.12 g, 20 mmol) were added to polyphosphoric acid to obtain a mixture. The mixture was gradually heated to 140° C. while stirring. When the TLC point plate reactant disappeared, the heat source was removed. After the reaction system was cooled, NaOH aqueous solution was added at the presence of ice water bath. A total of 5.2 g of Z10 was obtained by vacuum filtration, with a yield of 85%.

Z10 (4.6 g, 15 mmol), bis(pinacolato)diboron (2.03 g, 8 mmol), palladium acetate (0.11 g, 0.5 mmol), DPEPhos (0.54 g, 1 mmol), t-BuOK (7.28 g, 65 mmol) were weighed and dissolved into toluene to obtain a mixture. After purging the reaction vessel by nitrogenfor 5 times, the mixture was reacted completely while stirring at 110° C. for 1.5 h. After the reaction solution was cooled to room temperature, the reaction solution was layered with water, and the organic layer was obtained by extracting with ethyl acetate. The organic layer was dried and concentrated with anhydrous sodium sulfate, and purified by silica gel column to obtain Z11 with a total yield of 4.17 g and a yield of 61%.

Compound Z11 (3.64 g, 8 mmol) and carbazole (3 g, 18 mmol) were dissolved into anhydrous toluene to obtain a mixture. Sodium tert-butoxide (1.73 g, 18 mmol) and tris(dibenzylideneacetone)dipalladium (0.37 g, 0.4 mmol) were added into the mixture. After purging the reaction vessel by nitrogenfor three times, tri-tert-butylphosphine (0.4 mmol) was added into the mixture. The mixture was gradually heated to 80° C., while stirring for 12 hours, and the heat source was removed. After the reaction system was cooled, deionized water was added to separate the organic layer. The organic layer was extracted with ethyl acetate for three times, concentrated under reduced pressure, and purified on silica gel column chromatography to obtain 4.6 g of product C13, with a yield of 80%. MS: 718 [M⁺].

### Synthesis of Compound C23

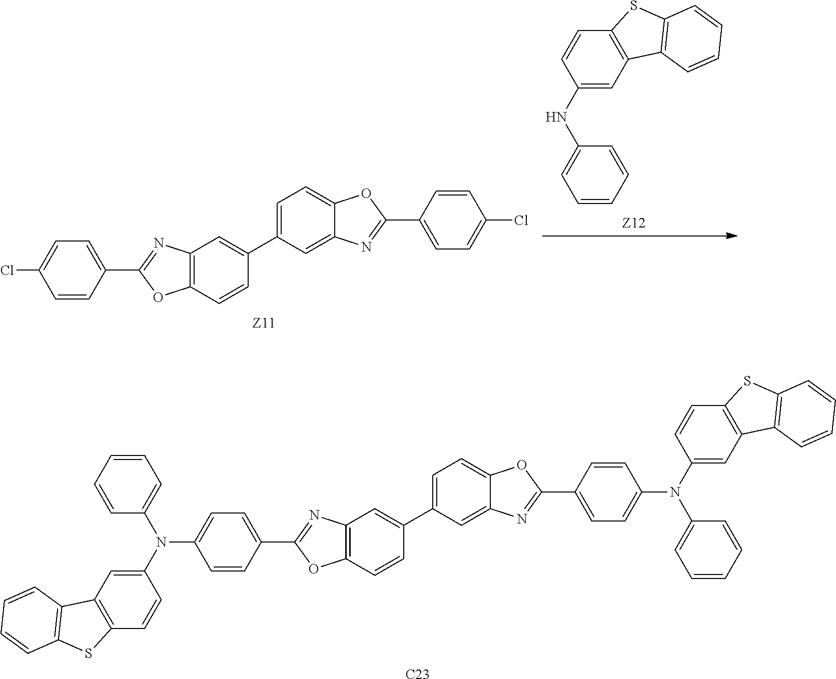

Compounds Z11 (4.56 g, 10 mmol) and Z12 (5.77 g, 21 mmol) were dissolved in anhydrous toluene to obtain a mixture. Sodium tert-butoxide (2 g, 21 mmol) and tris(dibenzylideneacetone)dipalladium (0.46 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 6.35 g of product C23 with a yield of 68%. MS: 934 [M⁺].

### Synthesis of Compound C25

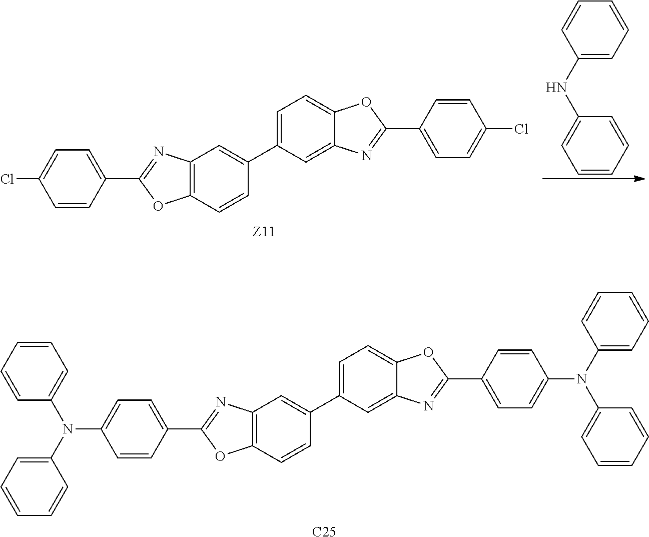

Compound Z11 (4.56 g, 10 mmol) and diphenylamine (2.54 g, 15 mmol) were dissolved in anhydrous toluene to obtain a mixture. Sodium tert-butoxide (1.44 g, 15 mmol) and tris(dibenzylideneacetone)dipalladium (0.19 g, 0.21 mmol) were added into the mixture. After purging the reaction vessel by nitrogenfor three times, tri-tert-butylphosphine (0.21 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 3.64 g of product C25 with a yield of 72%. MS: 722 [M⁺].

### Synthesis of Compound C26

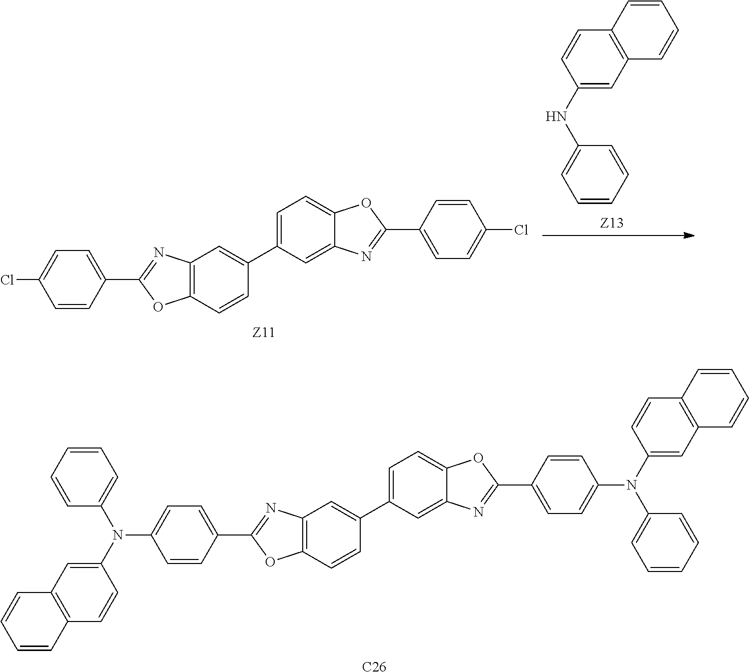 [SMILES: Clc1ccc(-c2nc3cc(-c4ccc5oc(-c6ccc(Cl)cc6)nc5c4)ccc3o2)cc1 ; c1ccc(Nc2ccc3ccccc3c2)cc1 ; c1ccc(N(c2ccc(-c3nc4cc(-c5ccc6oc(-c7ccc(N(c8ccccc8)c8ccc9ccccc9c8)cc7)nc6c5)ccc4o3)cc2)c2ccc3ccccc3c2)cc1 ; "C26"]

Compounds Z11 (4.56 g, 10 mmol) and Z13 (4.6 g, 21 mmol) were dissolved in anhydrous toluene to obtain a mixture. Sodium tert-butoxide (2 g, 21 mmol) and tris(dibenzylideneacetone)dipalladium (0.46 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 5.42 g of product C26 with a yield of 66%. MS: 822 [M⁺].

### Synthesis of compound C27

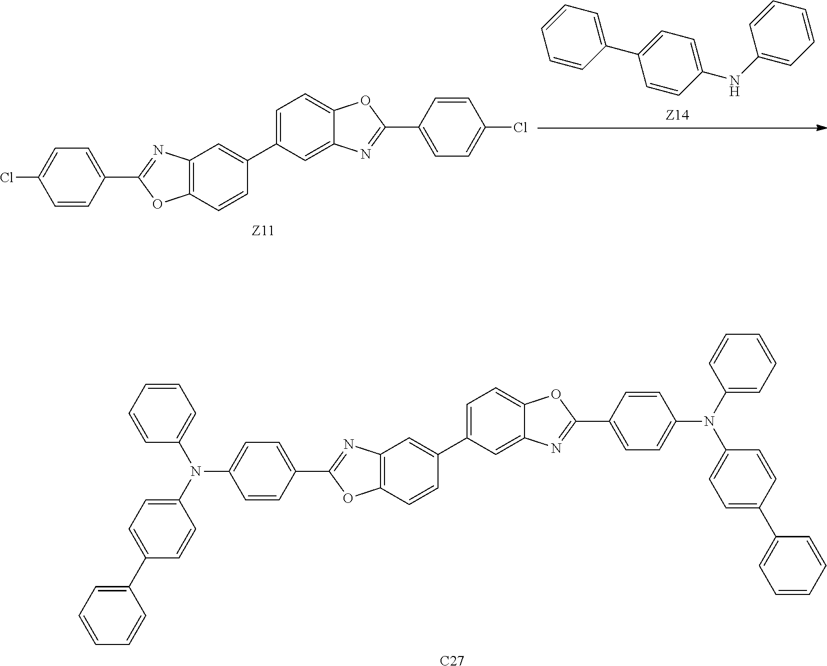 [SMILES: Clc1ccc(-c2nc3cc(-c4ccc5oc(-c6ccc(Cl)cc6)nc5c4)ccc3o2)cc1 ; c1ccc(Nc2ccc(-c3ccccc3)cc2)cc1 ; c1ccc(-c2ccc(N(c3ccccc3)c3ccc(-c4nc5cc(-c6ccc7oc(-c8ccc(N(c9ccccc9)c9ccc(-c%10ccccc%10)cc9)cc8)nc7c6)ccc5o4)cc3)cc2)cc1 ; "C27"]

Compounds Z11 (4.56 g, 10 mmol) and Z14 (5.14 g, 21 mmol) were dissolved in anhydrous toluene to obtain a mixture. Sodium tert-butoxide (2 g, 21 mmol) and tris(dibenzylideneacetone)dipalladium (0.45 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 7.08 g of product C27 with a yield of 81%. MS: 874 [M⁺].

### Synthesis of Compound C29

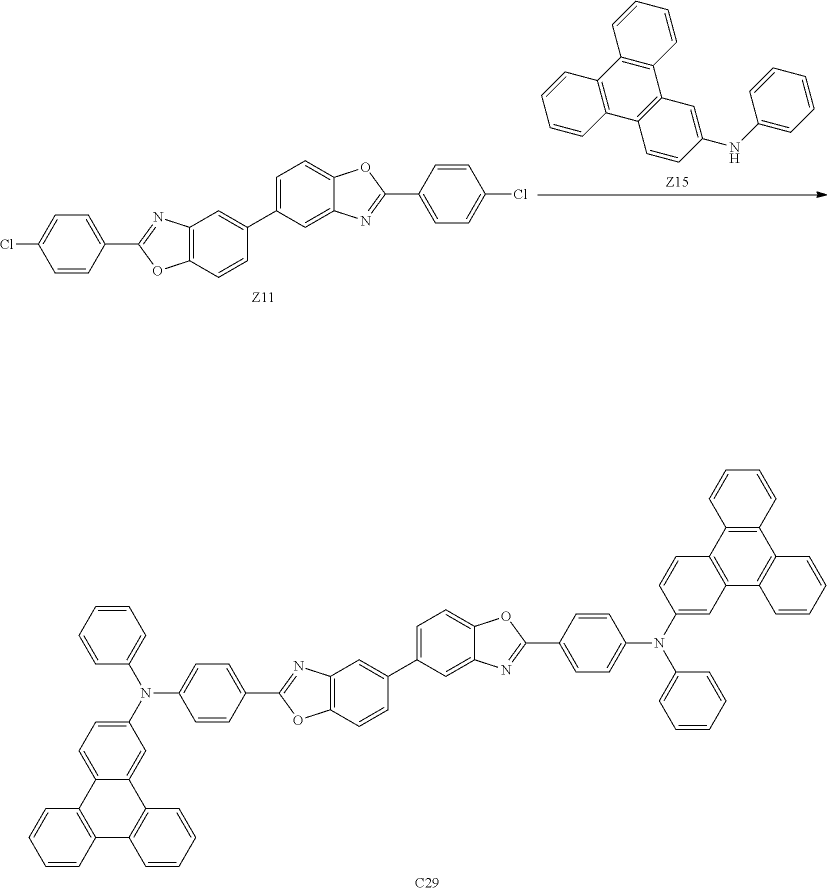 [SMILES: Clc1ccc(-c2nc3cc(-c4ccc5oc(-c6ccc(Cl)cc6)nc5c4)ccc3o2)cc1 ; c1ccc(Nc2ccc3c4ccccc4c4ccccc4c3c2)cc1 ; c1ccc(N(c2ccc(-c3nc4cc(-c5ccc6oc(-c7ccc(N(c8ccccc8)c8ccc9c%10ccccc%10c%10ccccc%10c9c8)cc7)nc6c5)ccc4o3)cc2)c2ccc3c4ccccc4c4ccccc4c3c2)cc1 ; "C29"]

Compounds Z11 (4.56 g, 10 mmol) and Z15 (6.7 g, 21 mmol) were dissolved in anhydrous toluene to obtain a mixture. Sodium tert-butoxide (2 g, 21 mmol) and tris(dibenzylideneacetone)dipalladium (0.45 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 5.82 g of product C29 with a yield of 57%. MS: 1022 [M⁺].

### Synthesis of Compound C33

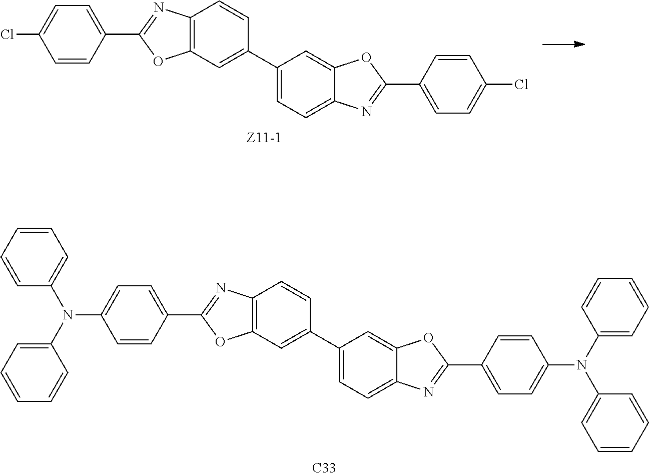 [SMILES: Clc1ccc(-c2nc3ccc(-c4ccc5nc(-c6ccc(Cl)cc6)oc5c4)cc3o2)cc1 ; c1ccc(N(c2ccccc2)c2ccc(-c3nc4ccc(-c5ccc6nc(-c7ccc(N(c8ccccc8)c8ccccc8)cc7)oc6c5)cc4o3)cc2)cc1 ; "C33"]

The synthesis steps of compound Z11-1 are similar to Z11.

Compound Z11-1 (4.56 g, 10 mmol) and diphenylamine (3.55 g, 21 mmol) were dissolved in anhydrous toluene to obtain a mixture. Sodium tert-butoxide (2 g, 21 mmol) and tris(dibenzylideneacetone)dipalladium (0.45 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 4.69 g of product C33 with a yield of 65%. MS: 722 [M⁺].

### Synthesis of Compound C37

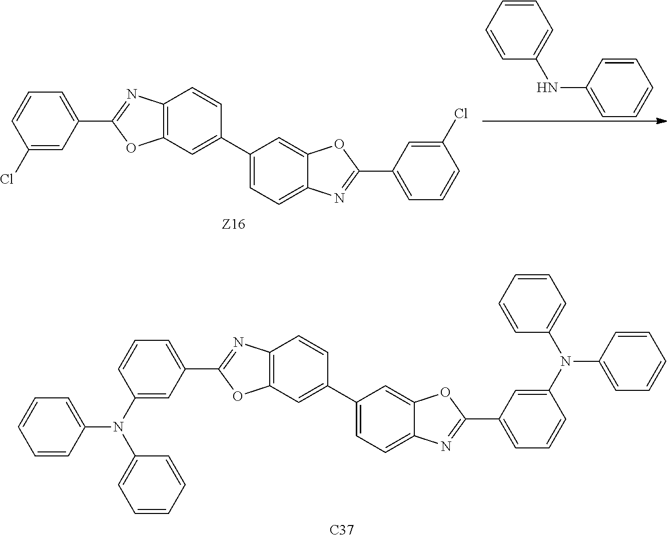 [SMILES: Clc1cccc(-c2nc3ccc(-c4ccc5nc(-c6cccc(Cl)c6)oc5c4)cc3o2)c1 ; c1ccc(Nc2ccccc2)cc1 ; c1ccc(N(c2ccccc2)c2cccc(-c3nc4ccc(-c5ccc6nc(-c7cccc(N(c8ccccc8)c8ccccc8)c7)oc6c5)cc4o3)c2)cc1 ; "C37"]

The synthesis steps of compound Z16 are similar to Z11.

Compound Z16 (4.56 g, 10 mmol) and diphenylamine (3.55 g, 21 mmol) were dissolved in anhydrous toluene to obtain a mixture. Sodium tert-butoxide (2 g, 21 mmol) and tris(dibenzylideneacetone)dipalladium (0.45 g, 0.5 mmol) were added into the mixture. After purging the reaction vessel by nitrogenfor three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 6.13 g of product C37 with a yield of 85%. MS: 722 [M⁺].

### Synthesis of Compound C57

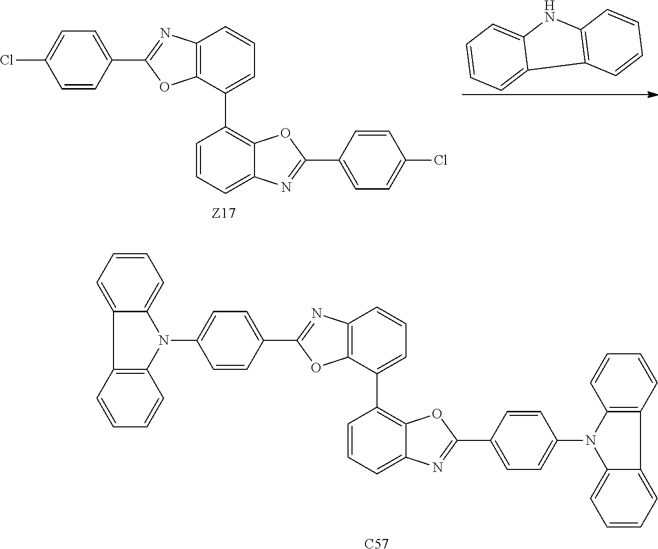 [SMILES: Clc1ccc(-c2nc3cccc(-c4cccc5nc(-c6ccc(Cl)cc6)oc45)c3o2)cc1 ; c1ccc2c(c1)[nH]c1ccccc12 ; c1cc(-c2cccc3nc(-c4ccc(-n5c6ccccc6c6ccccc65)cc4)oc23)c2oc(-c3ccc(-n4c5ccccc5c5ccccc54)cc3)nc2c1 ; "C57"]

The synthesis steps of compound Z17 are similar to Z11.

Compound Z17 (4.56 g, 10 mmol) and carbazole (3.5 g, 21 mmol) were dissolved in anhydrous toluene to obtain a mixture. sodium tert-butoxide (2 g, 21 mmol) and tris(dibenzylideneacetone)dipalladium (0.45 g, 0.5 mmol) were added. After purging the reaction vessel by nitrogen for three times, tri-tert-butylphosphine (0.5 mmol) was added into the mixture. The mixture was gradually heated to 80° C. while stirring for 12 hours, and the heat source was removed. After the reaction system was cooled, deionized water was added to separate the organic layer. The organic layer was extracted with ethyl acetate for three times, concentrated under reduced pressure, and then purified on silica gel column chromatography to obtain 5.24 g of product C57 with a yield of 73%. MS: 718 [M⁺].

### Calculation of Extinction Coefficient and Refractive Index

A compound was evaporated on a single crystal silicon by vacuum evaporation to form a thin film of 50 nm. The single crystal silicon was placed on the ellipsometer (ES-01) sample stage for test. The incident angle was 70°, and the test environment was atmospheric environment. The extinction coefficient (k) and refractive index (n) test results of the compound were fitted by the ellipsometer.

The results are shown in Table 1.

TABLE 1

| Compound | Extinction Coefficient @430 nm | Refractive Index @630 nm |
|---|---|---|
| C1 | 0 | 1.88 |
| C2 | 0 | 1.89 |
| C3 | 0 | 1.88 |
| C13 | 0 | 1.84 |
| C23 | 0.01 | 1.87 |
| C25 | 0.01 | 1.85 |
| C26 | 0.02 | 1.86 |
| C27 | 0.01 | 1.85 |
| C29 | 0.02 | 1.87 |
| C33 | 0.08 | 1.87 |
| C37 | 0 | 1.84 |
| C57 | 0 | 1.82 |
| Ref-1 | 0 | 1.76 |
| Ref-2 | 0 | 1.75 |
| Ref-3 | 0 | 1.78 |
| CBP | 0 | 1.74 |

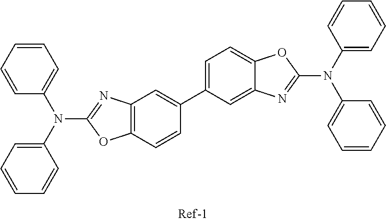 [SMILES: c1ccc(N(c2ccccc2)c2nc3cc(-c4ccc5oc(N(c6ccccc6)c6ccccc6)nc5c4)ccc3o2)cc1]

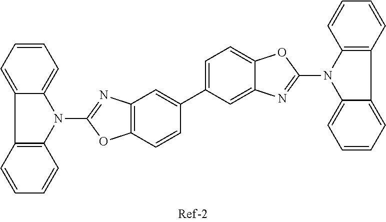 [SMILES: c1ccc2c(c1)c1ccccc1n2-c1nc2cc(-c3ccc4oc(-n5c6ccccc6c6ccccc65)nc4c3)ccc2o1]

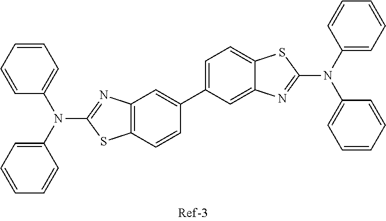 [SMILES: c1ccc(N(c2ccccc2)c2nc3cc(-c4ccc5sc(N(c6ccccc6)c6ccccc6)nc5c4)ccc3s2)cc1]

The compound of the present disclosure has a weak absorption in the visible light band and a higher absorption in the ultraviolet band, which is capable of resisting damage to the internal components of the device caused by high-energy external light. A higher refractive index may ensure better light extraction results.

### Preparation and Characterization of OLED Devices

The following is a detailed description of the preparation process using the aforementioned OLED device by specific embodiments. The structure of the OLED device is: ITO/Ag/ITO (anode)/HATCN/SFNFB/m-CP: Ir(p-ppy)₃/NaTzF₂/LiF/Mg: Ag/light extraction layer. The preparation steps are as follows.

After the ITO conductive glass anode layer was cleaned, the ITO conductive glass anode layer was ultrasonically cleaned with deionized water, acetone, and isopropanol for 15 minutes, and then treated in a plasma cleaner for 5 minutes to increase the electrode work function. A hole injection layer material HATCN was deposited on the ITO anode layer by vacuum evaporation, the thickness was 5 nm, and the deposition rate was 1 Å/s. A hole transport material SFNFB was deposited on the hole injection layer by vacuum evaporation, and the thickness was 80 nm. A light-emitting layer is deposited on the hole transport layer, m-CP is used as the host material, Ir(p-ppy)₃ is used as the doping material, the mass ratio of Ir(p-ppy)₃ and m-CP is 1:9, and the thickness is 30 nm. An electron transport material NaTzF₂ was deposited on the light-emitting layer by vacuum evaporation, and the thickness was 30 nm. An electron injection layer LiF was deposited on the electron transport layer by vacuum evaporation, the thickness was 1 nm, and the layer is an electron injection layer. A cathode Mg:Ag layer was deposited on the electron injection layer by vacuum evaporation, the doping ratio of Mg: Ag was 9:1, and the thickness was 15 nm. The compound C1 with a thickness of 60 nm was deposited on the cathode layer by vacuum evaporation.

Device example 2: The compound of the light extraction layer of the organic electroluminescent device was changed to C2.

Device example 3: The compound of the light extraction layer of the organic electroluminescent device was changed to C3.

Device example 4: The compound of the light extraction layer of the organic electroluminescent device was changed to C13.

Device example 5: The compound of the light extraction layer of the organic electroluminescent device was changed to C23.

Device example 6: The compound of the light extraction layer of the organic electroluminescent device was changed to C25.

Device example 7: The compound of the light extraction layer of the organic electroluminescent device was changed to C26.

Device example 8: The compound of the light extraction layer of the organic electroluminescent device was changed to C27.

Device example 9: The compound of the light extraction layer of the organic electroluminescent device was changed to C29.

Device example 10: The compound of the light extraction layer of the organic electroluminescent device was changed to C37.

Device example 11: The compound of the light extraction layer of the organic electroluminescent device was changed to C57.

Device example 12: The compound of the light extraction layer of the organic electroluminescent device was changed to C33.

Device comparison example 1: The compound of the light extraction layer of the organic electroluminescent device was changed to CBP.

Device comparison example 2: The compound of the light extraction layer of the organic electroluminescent device was changed to compound Ref-1.

Device comparison example 3: The compound of the light extraction layer of the organic electroluminescent device was changed to compound Ref-2.

Device comparison example 4: The compound of the light extraction layer of the organic electroluminescent device was changed to compound Ref-3.

The compounds involved in the device have the following structures.

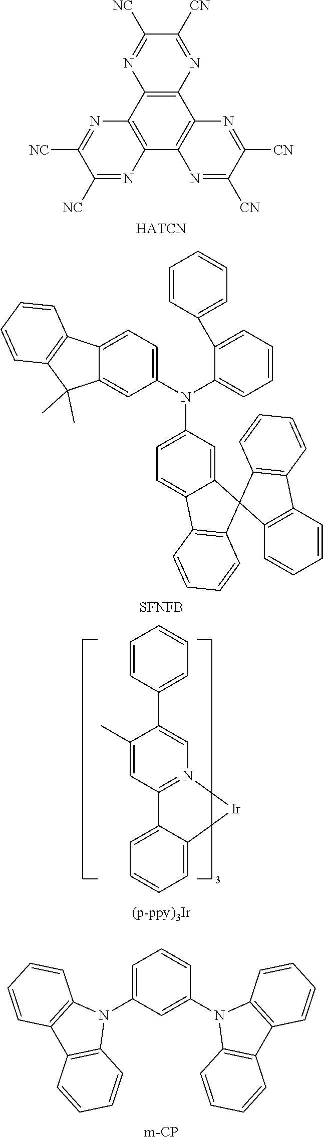

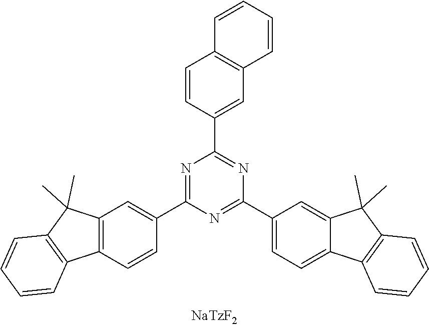 [SMILES: CC1(C)c2ccccc2-c2ccc(-c3nc(-c4ccc5c(c4)C(C)(C)c4ccccc4-5)nc(-c4ccc5ccccc5c4)n3)cc21]

TABLE 2

| | Compound of the | Luminous |
|---|---|---|
| No. | Light Extraction Layer | Efficiency |
| Device Example 1 | C1 | 1.18 |
| Device Example 2 | C2 | 1.19 |
| Device Example 3 | C3 | 1.17 |
| Device Example 4 | C13 | 1.12 |
| Device Example 5 | C23 | 1.16 |
| Device Example 6 | C25 | 1.13 |
| Device Example 7 | C26 | 1.15 |
| Device Example 8 | C27 | 1.14 |
| Device Example 9 | C29 | 1.15 |
| Device Example 10 | C37 | 1.12 |
| Device Example 11 | C57 | 1.1 |
| Device Example 12 | C33 | 1.16 |
| Comparative Example 1 | CBP | 1 |
| Comparative Example 2 | Ref-1 | 1.04 |
| Comparative Example 3 | Ref-2 | 1.02 |
| Comparative Example 4 | Ref-3 | 1.05 |

The luminous efficiency in Table 2 is the relative value obtained when the current density is 10 mA/cm². Seen from Table 2, compared to the comparative examples, the compounds of the present disclosure as the light extraction layer are capable of effectively improving the luminous efficiency of the organic electroluminescent device. This is because compared to the compounds Ref-1 to Ref-3, the introduction of phenyl group in the compound of the present disclosure significantly increases its conjugated structure, reducing the band gap of the compound and effectively improving its absorption in the wavelength range (<400 nm), which is beneficial for improving the refractive index of the compound and the luminous efficiency of the device.

The atomic radius of nitrogen atoms is larger than the atomic radius of oxygen atoms, and the structure

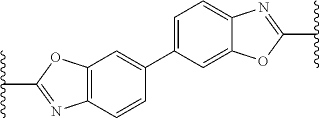 [SMILES: *c1nc2ccc(-c3ccc4nc(*)oc4c3)cc2o1]

has stronger electron withdrawing ability than the structure

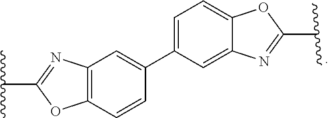 [SMILES: *c1nc2cc(-c3ccc4oc(*)nc4c3)ccc2o1]

Therefore, when the former is bonded to the aromatic amine group with strong electron donating ability, the electron push-pull is strong, which may cause some compounds to affect the light extraction. Therefore, the former is more suitable when paired with structures such as carbazole with slightly weaker electron donating ability. The latter because of the weak electron pulling, if it is bonded to a group such as carbazole, it will lead to weak absorption, and its bonding effect with the aromatic amine group will be better.

In addition, the structure with a five membered ring directly bonded to a nitrogen atom has poor thermal stability. For the compounds of the present disclosure, the thermal decomposition temperature (Td) of 1% weight loss can reach more than 400° C., and Td of 1% weight loss of compound C1 can even reach more than 490° C. Therefore, the compounds have a high tolerance for the evaporation rate.

The various technical features of the above embodiments can be combined as needed. To make the description concise, all possible combinations of the various technical features in the above embodiments have not been described. However, as long as there is no contradiction in the combination of these technical features, any of the combinations of the features should be considered within the scope of this specification.

The above only show some embodiments of the present disclosure, and description for them is more specific and detailed. However, it cannot be understood as a limitation on the scope of the disclosure. It should be pointed out that for those ordinary skilled in the art, any modifications and improvements can be made without departing from the concept of this disclosure, all of which fall within the scope of protection of this disclosure. Therefore, the scope of protection of this disclosure shall be based on the claims.

1. A compound for an organic electroluminescent device, having a structure as represented by formula (1): 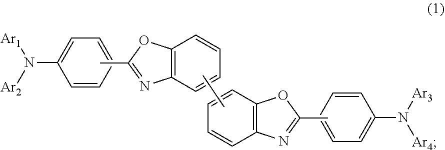
wherein,
Ar₁ to Ar₄ are independently selected from the group consisting of a substituted or unsubstituted aromatic group having 6 to 40 ring atoms, and a substituted or unsubstituted heteroaromatic group having 5 to 40 ring atoms; and
Ar₁ and Ar₂ form a ring or do not form a ring; and Ar₃ and Ar₄ form a ring or do not form a ring.

2. The compound for the organic electroluminescent device of claim 1, wherein Ar₁ to Ar₄ are independently selected from the group consisting of a substituted or unsubstituted aromatic group having 6 to 25 ring atoms, and a substituted or unsubstituted heteroaromatic group having 5 to 25 ring atoms.

3. The compound for the organic electroluminescent device of claim 1, wherein the compound is a center symmetric structure.

4. The compound for the organic electroluminescent device of claim 1, wherein the compound has a structure as represented by a following formula:

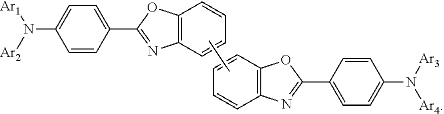

5. The compound for the organic electroluminescent device of claim 1, wherein the compound has a structure as represented by formula (2-1) or formula (2-2): 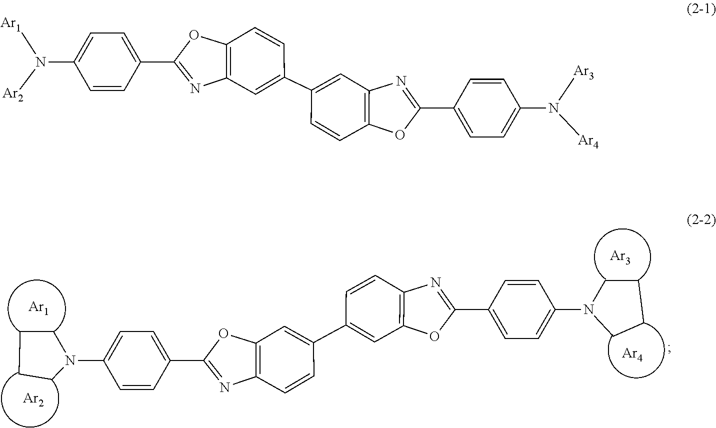
in the formula (2-1), Ar₁ and Ar₂ do not form a ring; and Ar₃ and Ar₄ do not form a ring.

6. The compound for the organic electroluminescent device of claim 1, wherein the compound has a structure as represented by any one of general formulae (2-3) to (2-6): 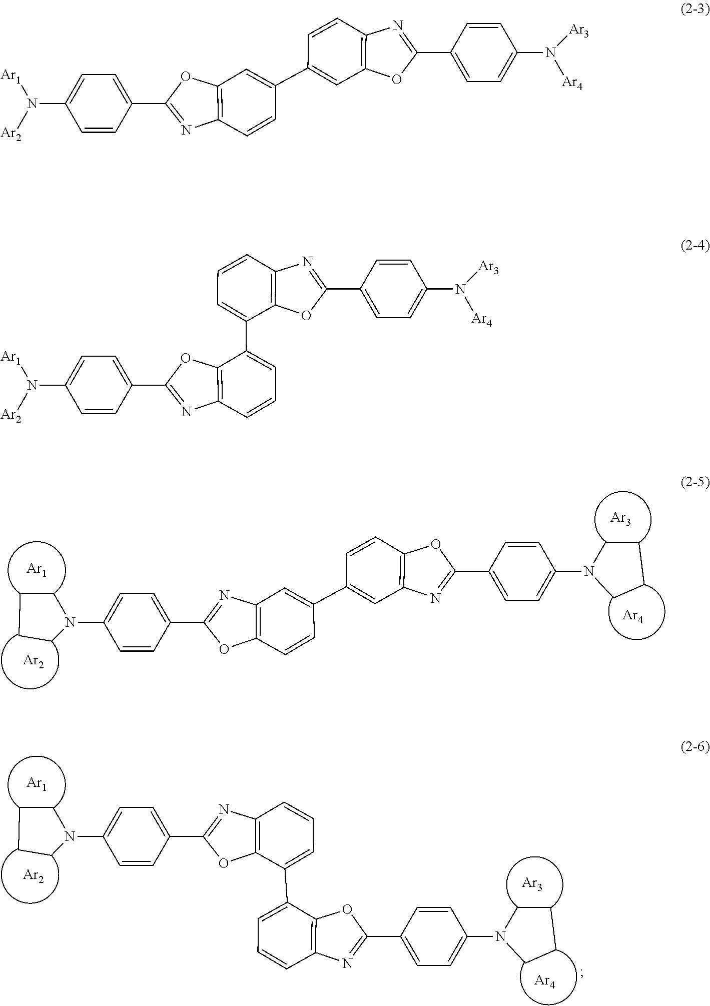
in the formulae (2-3) and (2-4), do not form a ring; and Ar₃ and Ar₄ do not form a ring.

7. The compound for the organic electroluminescent device of claim 1, wherein Ar₁ to Ar₄ are independently selected from the group consisting of following functional groups: 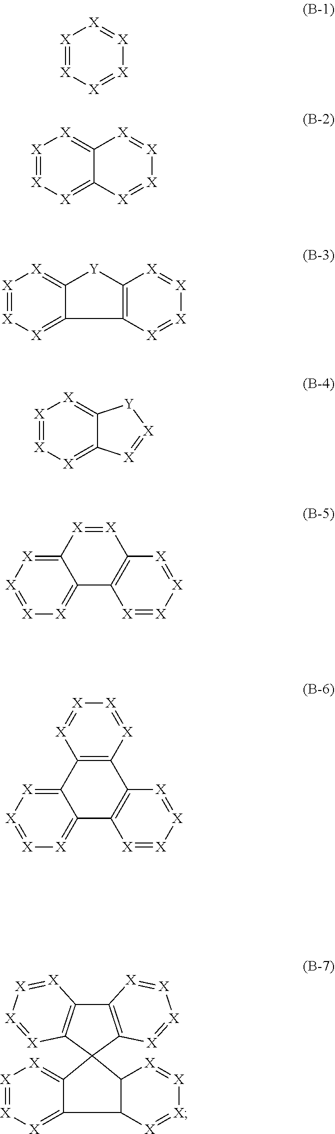
wherein, at each occurrence, X is independently selected from the group consisting of CR₁ and N;
at each occurrence, Y is independently selected from the group consisting of O, S, NR₂, and CR₃R₄;
at each occurrence, R₁ to R₄ are independently selected from the group consisting of —H, -D, a linear alkyl group having 1 to 20 carbon atoms, a linear alkoxy group having 1 to 20 carbon atoms, a linear thioalkoxy group having 1 to 20 carbon atoms, a branched or cyclic alkyl group having 3 to 20 carbon atoms, a branched or cyclic alkoxy group having 3 to 20 carbon atoms, a branched or cyclic thioalkoxy group having 3 to 20 carbon atoms, a silyl group, a ketone group having 1 to 20 carbon atoms, an alkoxycarbonyl group having 2 to 20 carbon atoms, an aryloxycarbonyl group having 7 to 20 carbon atoms, a cyano group, a carbamoyl group, a haloformyl group, a formyl group, an isocyano group, an isocyanate group, a thiocyanate group, an isothiocyanate group, a hydroxyl aroup, a nitro group, an amino group, —CF₃, —Cl, —Br, —F, a crosslinkable group, a substituted or unsubstituted aromatic group having 5 to 60 ring atoms, a substituted or unsubstituted heteroaromatic group having 5 to 60 ring atoms, an aryloxy group having 5 to 60 ring atoms, a heteroaryloxy group having 5 to 60 ring atoms, and any combination of the groups.

8. The compound for the organic electroluminescent device of claim 7, wherein at each occurrence, R₁ to R₄ are independently selected from the group consisting of -D, a linear alkyl group having 1 to 10 carbon atoms, a branched or cyclic alkyl group having 3 to 10 carbon atoms, a cyano group, a nitro group, —CF₃, —Cl, —Br, —F, a substituted or unsubstituted aromatic group having 6 to 30 ring atoms, and a substituted unsubstituted heteroaromatic group having 5 to 30 ring atoms, and any combination of the groups.

9. The compound for the organic electroluminescent device of claim 7, wherein at each occurrence, R₁ to R₄ are independently selected from the group consisting of —H, methyl, tertbutyl, phenyl, naphthyl and pyridinyl.

10. The compound for the organic electroluminescent device of claim 7, wherein Ar₁ to Ar₄ are independently selected from the group consisting of phenyl, biphenyl, terphenyl, naphthyl, phenanthryl, a triphenylenyl group, a spirocyclic group, pyridinyl, pyrimidinyl, triazinyl, quinolyl, isoquinolyl, dibenzofuranyl, dibenzothiophenyl, fluorenyl, 9,9-dimethylfluorenyl, carbazolyl, 9-phenyl-9-carbazolyl, benzofuranyl, benzothiophenyl, benzoxazolyl, benzothiazolyl, and any combination of the groups.

11. The compound for the organic electroluminescent device of claim 5, wherein in condition of the compound being selected from the formula (2-1), Ar₁ to Ar₄ are independently selected from the group consisting of following groups: 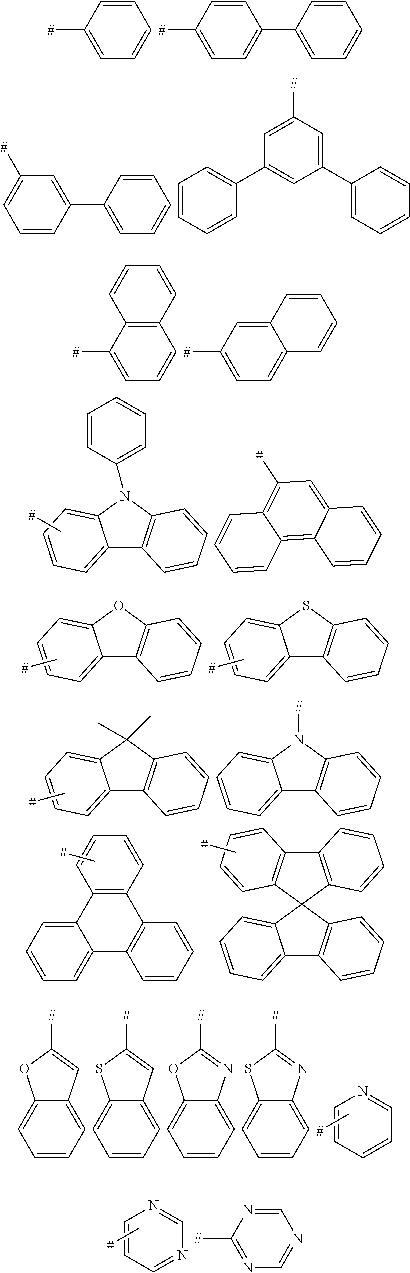
wherein “#” represents a connecting site; and
in condition of the compound being selected from the formulae (2-2), Ar₁ to Ar₄ are independently selected from the group consisting of following groups: 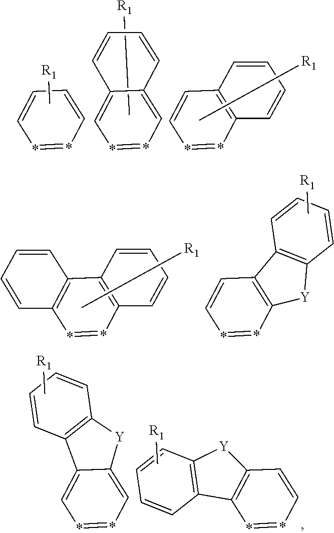
wherein “*” represents a fused site.

12. The compound for the organic electroluminescent device of claim 6, wherein in condition of the compound is selected from the group consisting of the formulae (2-5) and (2-6), Ar₁ to Ar₄ are independently selected from the group consisting of following groups: 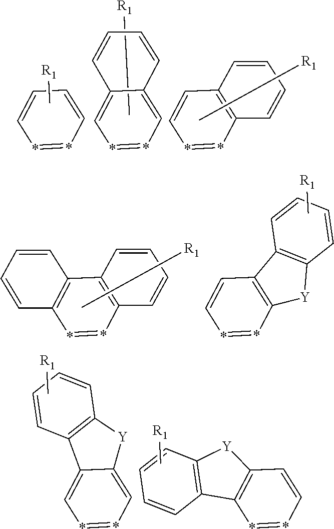
wherein “*” represents a fused site; and
in condition of the compound being selected from the formulae (2-3) and (2-4), Ar₁ to Ar₄ are independently selected from the group consisting of following groups: 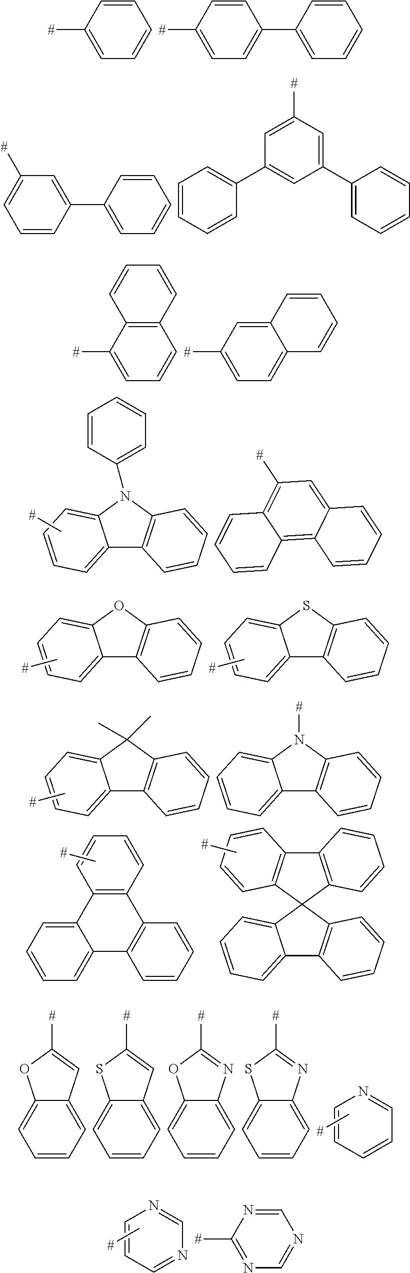
wherein “#” represents a connecting site.

13. The compound for the organic electroluminescent device of claim 1, wherein the compound is selected from the group consisting of following structures:

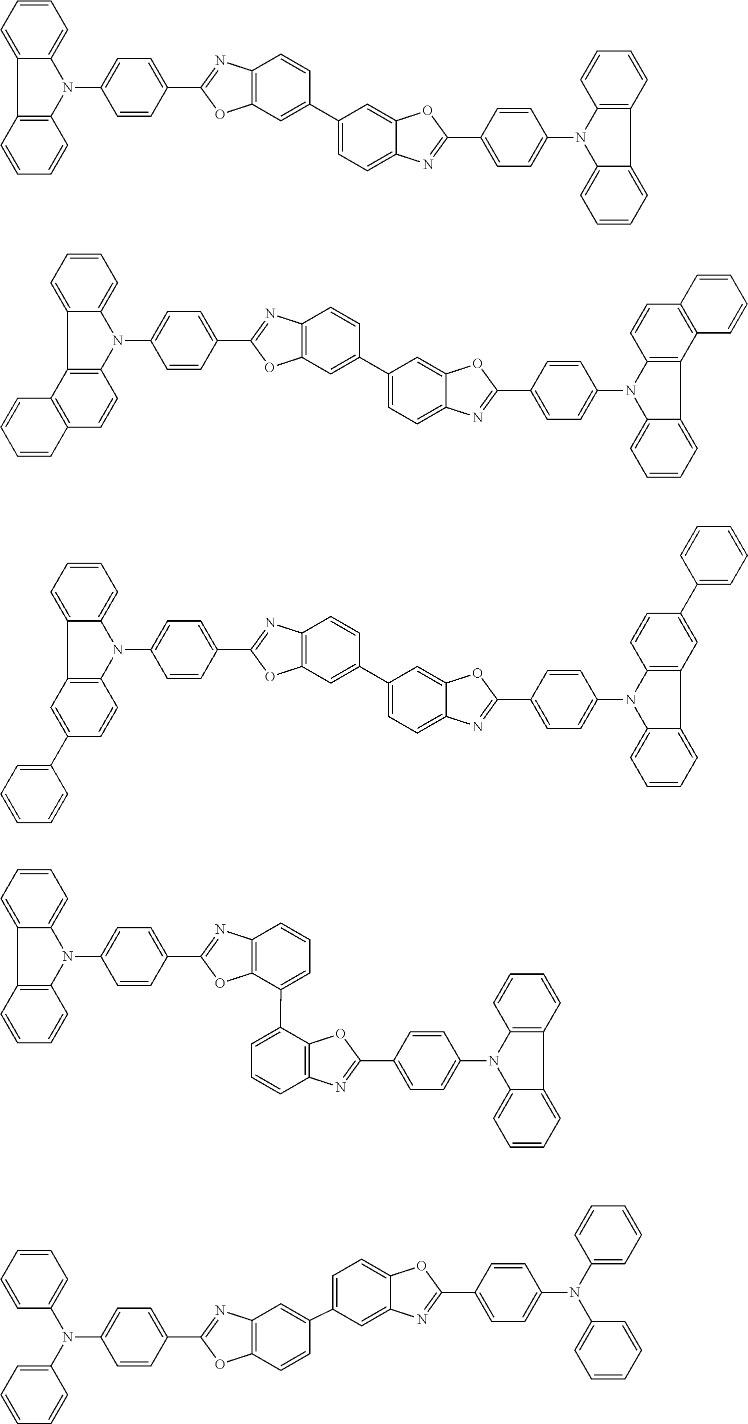

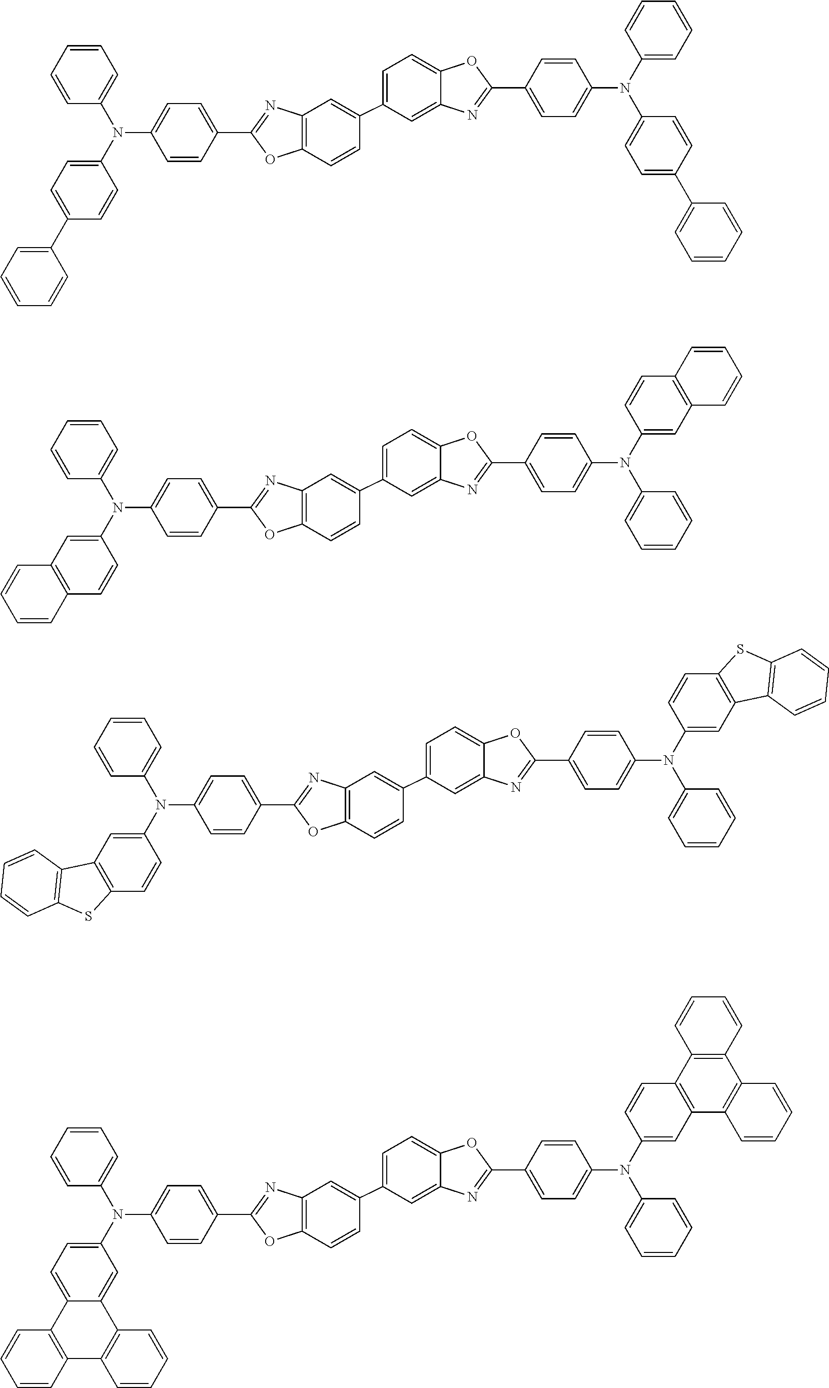 [SMILES: c1ccc(-c2ccc(N(c3ccccc3)c3ccc(-c4nc5cc(-c6ccc7oc(-c8ccc(N(c9ccccc9)c9ccc(-c%10ccccc%10)cc9)cc8)nc7c6)ccc5o4)cc3)cc2)cc1 ; c1ccc(N(c2ccc(-c3nc4cc(-c5ccc6oc(-c7ccc(N(c8ccccc8)c8ccc9ccccc9c8)cc7)nc6c5)ccc4o3)cc2)c2ccc3ccccc3c2)cc1 ; c1ccc(N(c2ccc(-c3nc4cc(-c5ccc6oc(-c7ccc(N(c8ccccc8)c8ccc9sc%10ccccc%10c9c8)cc7)nc6c5)ccc4o3)cc2)c2ccc3sc4ccccc4c3c2)cc1 ; c1ccc(N(c2ccc(-c3nc4cc(-c5ccc6oc(-c7ccc(N(c8ccccc8)c8ccc9c%10ccccc%10c%10ccccc%10c9c8)cc7)nc6c5)ccc4o3)cc2)c2ccc3c4ccccc4c4ccccc4c3c2)cc1]

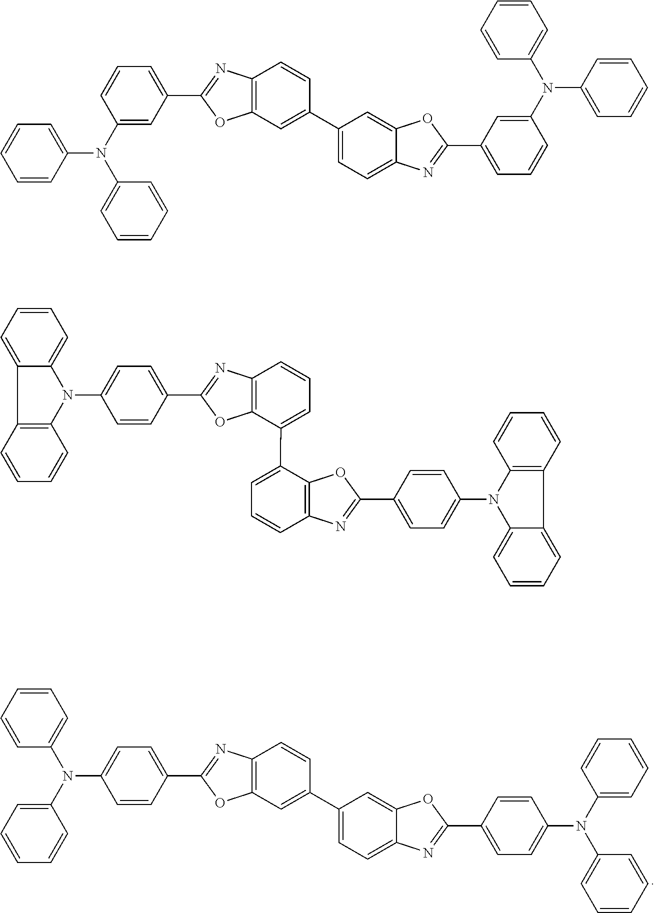 [SMILES: c1ccc(N(c2ccccc2)c2cccc(-c3nc4ccc(-c5ccc6nc(-c7cccc(N(c8ccccc8)c8ccccc8)c7)oc6c5)cc4o3)c2)cc1 ; c1cc(-c2cccc3nc(-c4ccc(-n5c6ccccc6c6ccccc65)cc4)oc23)c2oc(-c3ccc(-n4c5ccccc5c5ccccc54)cc3)nc2c1 ; c1ccc(N(c2ccccc2)c2ccc(-c3nc4ccc(-c5ccc6nc(-c7ccc(N(c8ccccc8)c8ccccc8)cc7)oc6c5)cc4o3)cc2)cc1]

14. A mixture, wherein the mixture comprises a compound and an organic functional material, the organic functional material is selected from one or more of a hole injection material, a hole transport material, an electron transport material, an electron injection material, an electron blocking material, a hole blocking material, a luminescent material, and a host material, wherein the compound has a structure as represented by formula (1): 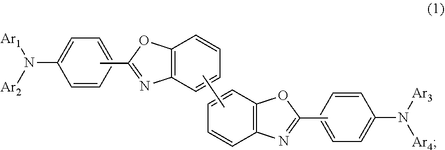
wherein,
Ar₁ to Ar₄ are independently selected from the group consisting of a substituted or unsubstituted aromatic group having 6 to 40 ring atoms, and a substituted or unsubstituted heteroaromatic group having 5 to 40 ring atoms; and
Ar₁ and Ar₂ form a ring or do not form a ring; and Ar₃ and Ar₄ form a ring or do not form a ring.

15. (canceled)

16. An organic electroluminescent device, comprising two electrodes, one or more organic functional layers disposed between the two electrodes, and a light extraction layer disposed on a surface of one of the two electrodes away from the organic functional layer, wherein a material of the light extraction layer comprises a compound, wherein the compound has a structure as represented by formula (1): 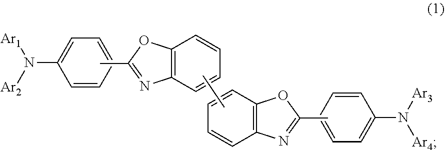
wherein,
Ar₁ to Ar₄ are independently selected from the group consisting of a substituted or unsubstituted aromatic group having 6 to 40 ring atoms, and a substituted or unsubstituted heteroaromatic group having 5 to 40 ring atoms; and
Ar₁ and Ar₂ form a ring or do not form a ring; and Ar₃ and Ar₄ form a ring or do not form a ring.

17. The organic electroluminescent device of claim 16, wherein the organic functional layer comprises a light-emitting layer, and a material of the light-emitting layer has following structure: 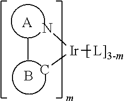
wherein m is selected from 1, 2, or 3;
ring A is selected from a substituted or unsubstituted N-containing heteroaromatic group having 5 to 30 ring atoms;
ring B is selected from a substituted or unsubstituted aromatic group or a heteroaromatic group having 6-30 ring atoms; and
L is a monovalent anionic organic ligand.

18. The organic electroluminescent device of claim 17, wherein the material of the light-emitting layer is selected from the group consisting of following structures: 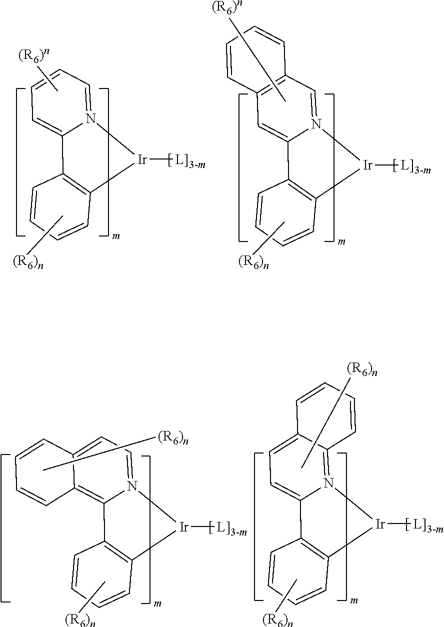
wherein,
at each occurrence, R₆ is independently selected from the group consisting of —H, -D, a linear alkyl group having 1 to 10 carbon atoms, a branched or cyclic alkyl group having 3 to 10 carbon atoms, a cyano group, a nitro group, —CF₃, —OCF₃, —Cl, —Br, —F, an aromatic group having 6 to 20 ring atoms, a heteroaromatic group having 5 to 20 ring atoms, and any combination of the groups; and
at each occurrence, n is independently selected from integers of 0 to 5.

19. The organic electroluminescent device of claim 18, wherein the material of the light-emitting layer is selected from following structure: 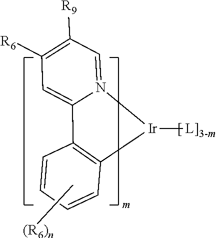
wherein at each occurrence, R₉ is independently selected from the group consisting of —H, -D, a linear alkyl group having 1 to 10 carbon atoms, a branched or cyclic alkyl group having 3 to 10 carbon atoms, a cyano group, a nitro group, —CF₃, —OCF₃, —Cl, —Br, —F, an aromatic group having 6 to 20 ring atoms, a heteroaromatic group having 5 to 20 ring atoms, and any combination of the groups.

20. The organic electroluminescent device of claim 19, wherein at each occurrence, R₉ is independently selected from the group consisting of phenyl, methyl, and deuterated methyl.
